(19) United States
(12) Patent Application Publication
(10) Pub. No.: US 2014/0147556 A1
(43) Pub. Date: May 29, 2014

(54) EDIBLE OR INHALABLE COMPOSITIONS HAVING ANTIBODIES AND METHODS OF USE

(71) Applicant: ELWHA LLC, Bellevue, WA (US)

(72) Inventors: Maxime J. J. Bilet, Seattle, WA (US); Ilya Fushman, Palo Alto, CA (US); Roderick A. Hyde, Redmond, WA (US); Jordin T. Kare, Seattle, WA (US); Vinod Khosla, Menlo Park, CA (US); David Mann, Menlo Park, CA (US); Nathan P. Myhrvold, Medina, WA (US); Clarence T. Tegreene, Mercer Island, WA (US); Lowell L. Wood, JR., Bellevue, WA (US); Christopher C. Young, Seattle, WA (US)

(73) Assignee: Elwha LLC, Bellevue, WA (US)

(21) Appl. No.: 13/686,741

(22) Filed: Nov. 27, 2012

(51) Int. Cl. C07K 16/28; A23L 1/22

(52) U.S. Cl. CPC C07K 16/28; A23L 1/22075
USPC 426/72; 530/389.1; 530/388.22; 530/387.3; 426/650; 426/648

(57) ABSTRACT

The present technology provides, in one aspect, an edible or inhalable composition including an isolated “antagonist ligand” or an isolated “antagonist antibody” or an antigen-binding fragment thereof, which specifically binds a taste receptor ligand or an olfactory receptor ligand. Foods and intranasal formulations containing the above-described compositions are described, as are methods of use.

### BACKGROUND

People generally prefer good tasting foods and pleasant smelling surroundings. Many foods such as candy, although virtually devoid of nutrition, are eagerly consumed because they taste good. Alternatively, some foods or edible substances are nutritious yet generally shunned by consumers because they taste bland or worse. As the global population grows so does the collective challenge of feeding and nourishing so many people. To do so, it will be helpful to more efficiently utilize an increasing range of edible and nutritious feedstocks, including bland and bad tasting substances, by making them more flavorful and enticing to eat. More effective ways of flavoring such foods are needed.

People are generally sensitive to bad odor and prefer not to smell such odors. Air fresheners, for example, mask odors by overwhelming the local environment with perfumes. When air fresheners are placed in a bad smelling room, they operate by increasing the airborne concentration of pleasant smelling odorants (e.g., perfume-like molecules) relative to bad smelling odorants until occupants of the room primarily smell the perfume rather than the odor. There are numerous disadvantages to this approach. Air fresheners are inefficient because they treat entire volumes of space, such as a room. Sometimes they over-treat and can be difficult to control. Air fresheners subject everyone within a treated room to a perfume smell, regardless of whether some people may be offended or allergic to that perfume. More personal and effective ways of combating bad smells are needed.

### SUMMARY

According to one exemplary embodiment, the present technology provides an edible composition including an isolated “antagonist ligand” or an isolated “antagonist antibody” or an antigen-binding fragment thereof, which specifically binds a taste receptor ligand.

According to another exemplary embodiment, the present technology provides an edible composition including an isolated bispecific “agonist antibody” or a bispecific antigen-binding fragment thereof, which specifically binds more than one epitope on a taste receptor and activates the taste receptor upon binding.

According to a further exemplary embodiment, the present technology provides a food item or beverage including any of the edible compositions described herein.

According to another exemplary embodiment, the present technology provides a method for modulating the flavor of food, comprising contacting the food with an effective amount of any of the edible compositions described herein.

According to another exemplary embodiment, the present technology provides a composition for intranasal administration including an isolated “antagonist ligand,” an isolated “antagonist antibody” or an antigen-binding fragment thereof, which specifically binds an olfactory receptor ligand.

The foregoing is a summary and thus by necessity contains simplifications, generalizations and omissions of detail. Consequently, those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein.

### BRIEF DESCRIPTION OF THE DRAWINGS

The skilled artisan will understand that the drawings primarily are for illustrative purposes and are not intended to limit the scope of the inventive subject matter described herein.

FIG. 1 shows the amino acid sequence (SEQ ID NO:1) of TAS1R1.

FIG. 2 shows the amino acid sequence (SEQ ID NO:2) of TAS1R2.

FIG. 3 shows the amino acid sequence (SEQ ID NO:3) of TAS1R3.

FIG. 4 shows the amino acid sequence (SEQ ID NO:4) of TAS2R1.

FIG. 5 shows the amino acid sequence (SEQ ID NO:5) of TAS2R3.

FIG. 6 shows the amino acid sequence (SEQ ID NO:6) of TAS2R4.

FIG. 7 shows the amino acid sequence (SEQ ID NO:7) of TAS2R5.

FIG. 8 shows the amino acid sequence (SEQ ID NO:8) of TAS2R7.

FIG. 9 shows the amino acid sequence (SEQ ID NO:9) of TAS2R8.

FIG. 10 shows the amino acid sequence (SEQ ID NO:10) of TAS2R9.

FIG. 11 shows the amino acid sequence (SEQ ID NO:11) of TAS2R10.

FIG. 12 shows the amino acid sequence (SEQ ID NO:12) of TAS2R13.

FIG. 13 shows the amino acid sequence (SEQ ID NO:13) of TAS2R14.

FIG. 14 shows the amino acid sequence (SEQ ID NO:14) of TAS2R16.

FIG. 15 shows the amino acid sequence (SEQ ID NO:15) of TAS2R19.

FIG. 16 shows the amino acid sequence (SEQ ID NO:16) of TAS2R20.

FIG. 17 shows the amino acid sequence (SEQ ID NO:17) of TAS2R30.

FIG. 18 shows the amino acid sequence (SEQ ID NO:18) of TAS2R31.

FIG. 19 shows the amino acid sequence (SEQ ID NO:19) of TAS2R38.

FIG. 20 shows the amino acid sequence (SEQ ID NO:20) of TAS2R39.

FIG. 21 shows the amino acid sequence (SEQ ID NO:21) of TAS2R40.

FIG. 22 shows the amino acid sequence (SEQ ID NO:22) of TAS2R41.

FIG. 23 shows the amino acid sequence (SEQ ID NO:23) of TAS2R42.

FIG. 24 shows the amino acid sequence (SEQ ID NO:24) of TAS2R43.

FIG. 25 shows the amino acid sequence (SEQ ID NO:25) of TAS2R44.

FIG. 26 shows the amino acid sequence (SEQ ID NO:26) of TAS2R45.

FIG. 27 shows the amino acid sequence (SEQ ID NO:27) of TAS2R46.

FIG. 28 shows the amino acid sequence (SEQ ID NO:28) of TAS2R49.

FIG. 29 shows the amino acid sequence (SEQ ID NO:29) of TAS2R50.

FIG. 30 shows the amino acid sequence (SEQ ID NO:30) of TAS2R60.

FIG. 31 shows the amino acid sequence (SEQ ID NO:31) of HCN1.

FIG. 32 shows the amino acid sequence (SEQ ID NO:32) of HCN4.

FIG. 33 shows the amino acid sequence (SEQ ID NO:33) of ACCN1.

FIG. 34 shows the amino acid sequence (SEQ ID NO:34) of TASK-1.

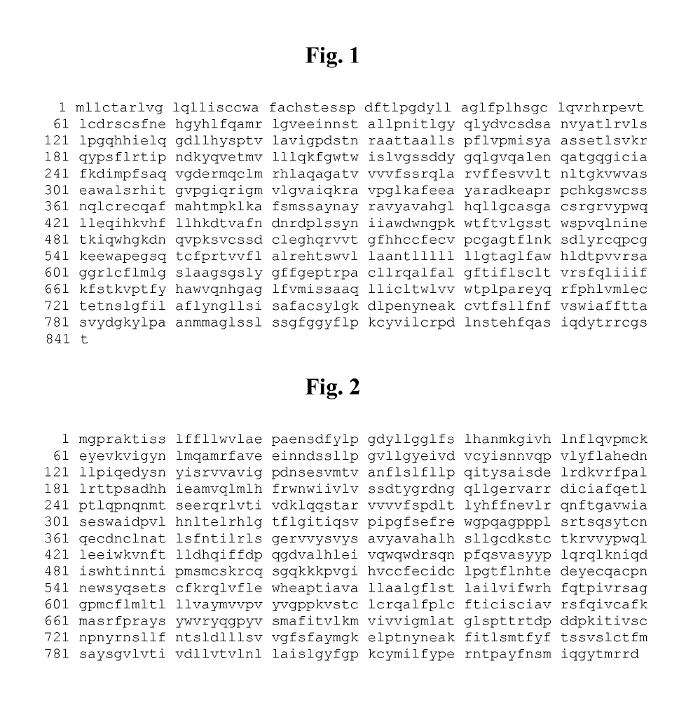

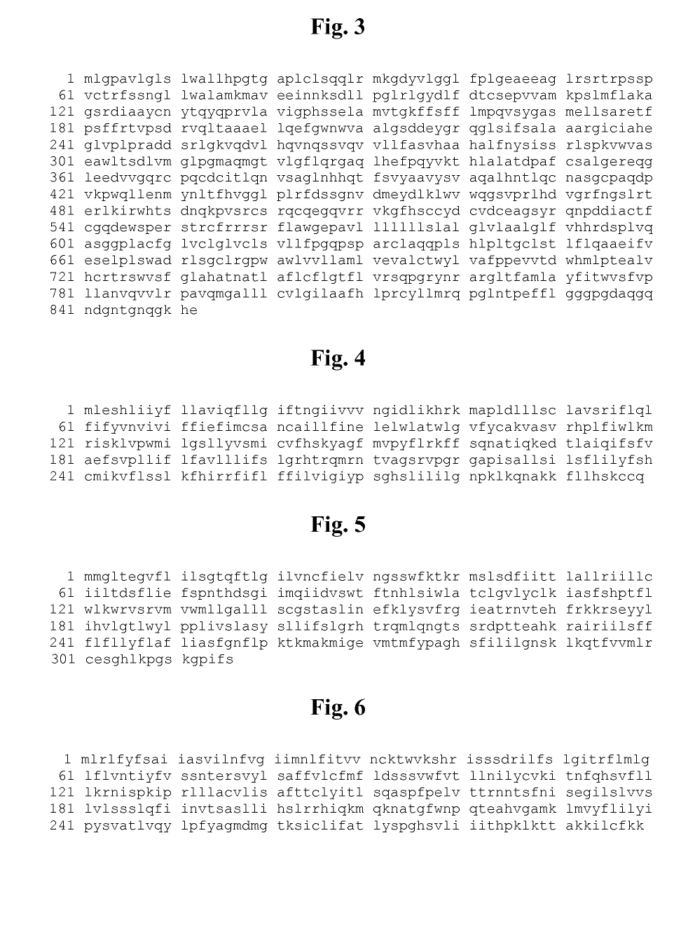

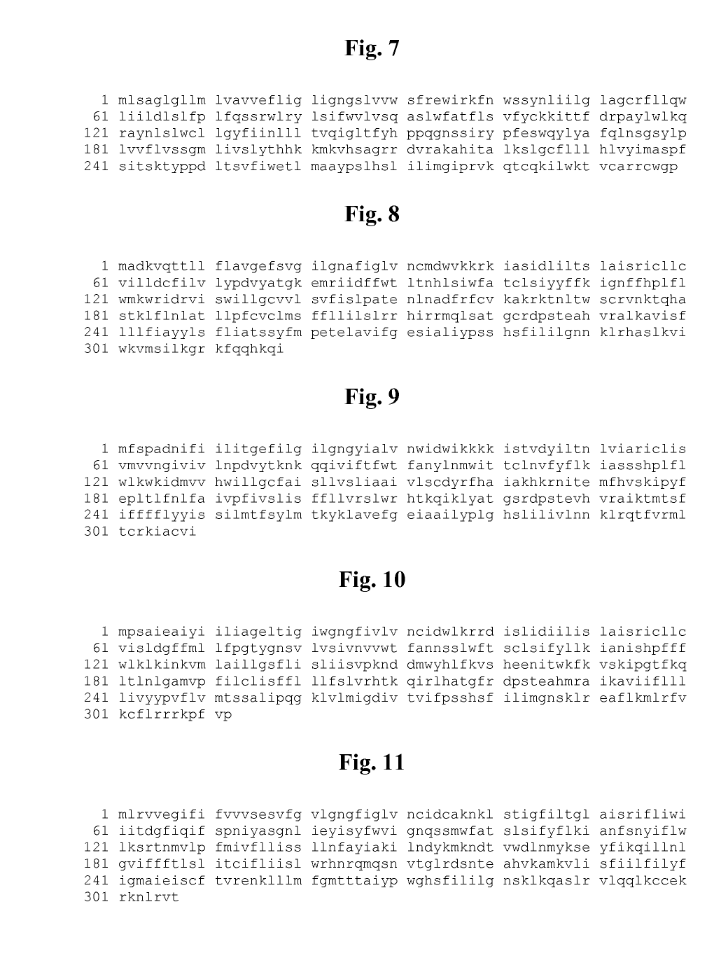

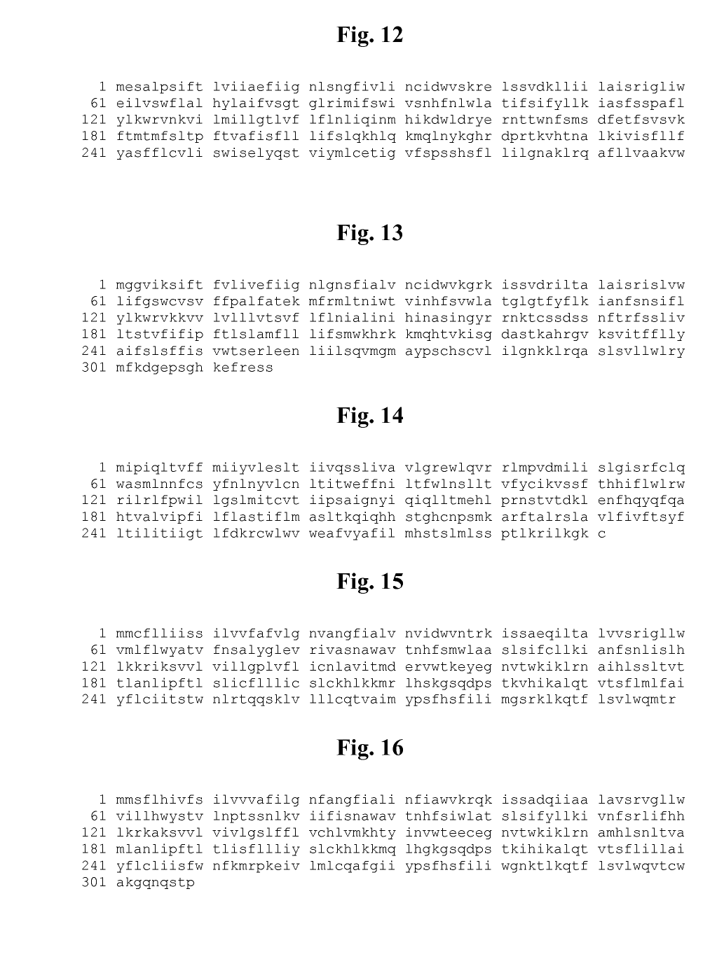

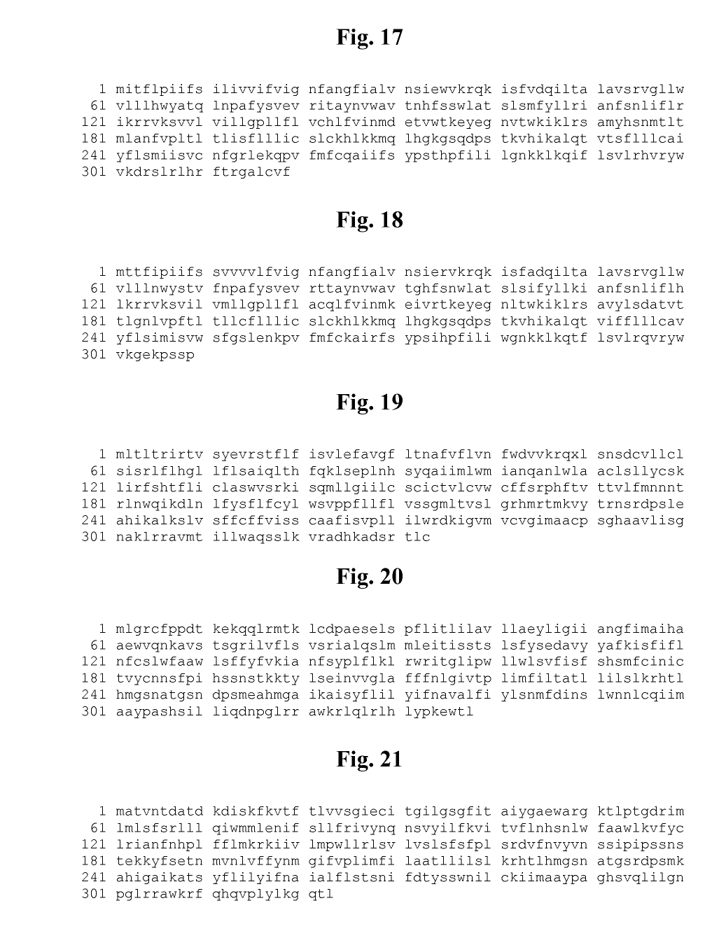

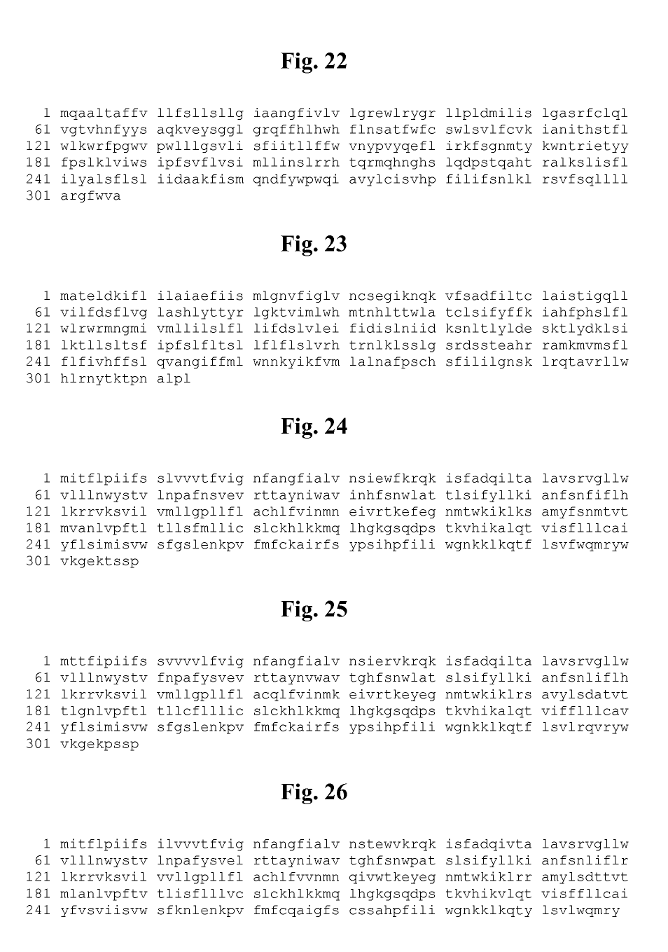

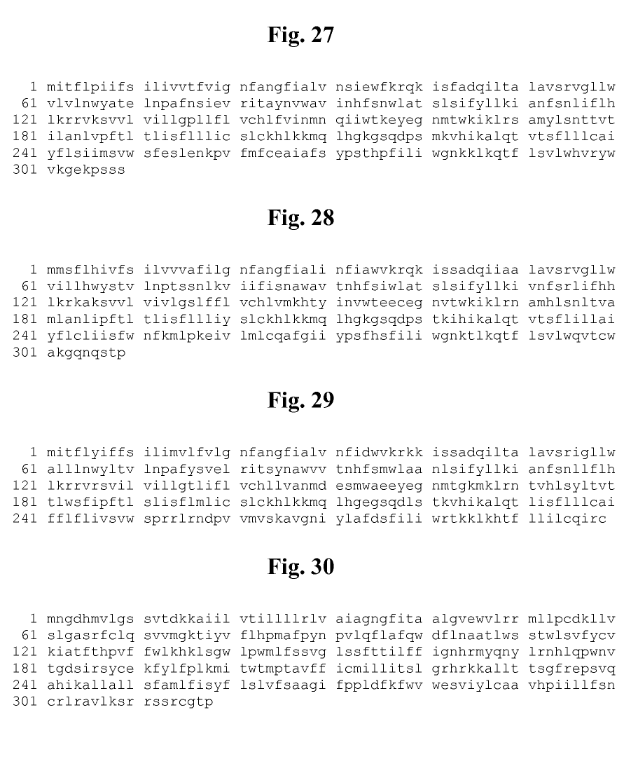

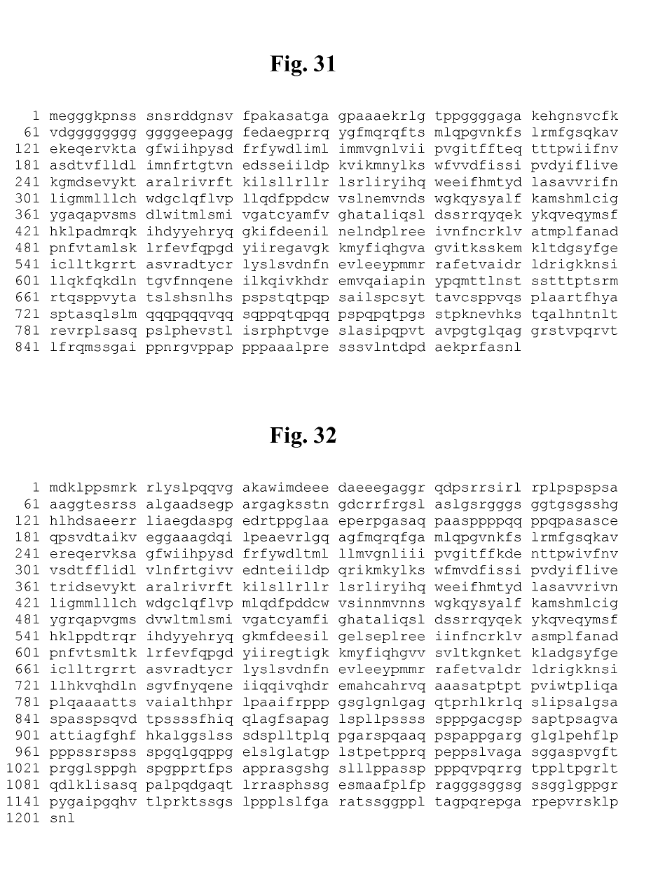

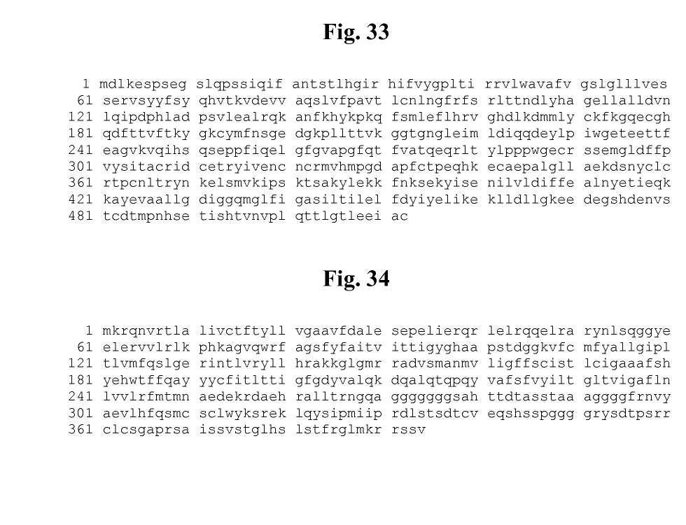

### DETAILED DESCRIPTION

The illustrative embodiments described in the detailed description, drawings, and claims are not meant to be limiting. Other embodiments may be utilized, and other changes may be made, without departing from the spirit or scope of the subject matter presented here.

The technology is described herein using several definitions, as set forth throughout the specification.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The technology described herein provides compositions and methods for activating and deactivating the sensation of taste and smell. As such, the edible compositions described herein can be added to foods as a seasoning or incorporated into foods during processing. Alternatively, the inhalable (e.g., intranasal) compositions described herein can be administered into the nose to alter a person's perception of smell.

In some embodiments, the edible/inhalable compositions described herein mimic the taste of flavorful foods or aromas by using an isolated “agonist antibody” or an antigen-binding fragment thereof, which specifically binds one or more taste/olfactory receptors. The agonist antibody is a “primary antibody” that targets and specifically binds one or more taste/olfactory receptors. Upon binding the taste/olfactory receptor, such as a sweet taste receptor, the agonist antibody activates the sweet taste receptor causing the sensation of sweet flavor in the consumer. Accordingly, the edible/inhalable composition can include numerous agonist antibodies that bind numerous taste/olfactory receptors and produce the sensation of “scores of flavors” or “scores of aromas” in the consumer.

The edible compositions described herein, for example, containing an agonist antibody or fragment thereof, can formulated as a seasoning that can be added to existing foods. Alternatively, the edible compositions described herein containing an agonist antibody or fragment thereof, can be incorporated into foods during processing to provide flavor to otherwise bland foods.

Additionally, the flavors or aromas provided by the agonist antibodies described herein can be deactivated (i.e., turned-off) by the addition (e.g., consumption or inhalation) of an edible/inhalable composition that includes an isolated “antagonist ligand” or an isolated “antagonist antibody” or an antigen-binding fragment thereof. These antagonists deactivate the sensation of a particular taste or smell, for example, e.g., such as a sweet taste, by specifically binding either (i) an agonist (e.g., primary) antibody or antigen-binding fragment described herein to bind to a sweet taste receptor, or (ii) competitively binding, and thus intercepting, one or more agonist ligands, such as sucrose, that would otherwise activate a sweet taste receptor. Thus, the antagonist antibody may be a “secondary antibody” or “binding peptide” that targets and specifically binds the “agonist antibody” (e.g., “primary antibody”) rather than a taste/olfactory receptors. As such, edible compositions that include an isolated antagonist ligand or an isolated antagonist antibody can be used to deactivate, or turn off, the sensation of particular flavors or aromas. Such edible compositions that include an isolated antagonist ligand or an isolated antagonist antibody, or an antigen-binding fragment thereof, can be added to flavorful foods or bad tasting foods to make them less flavorful.

In some embodiments, the antagonist ligand is a small molecule having a molecular weight less than 500 (atomic mass units) that binds any of the agonist antibodies described herein and thereby deactivates or inhibits the binding affinity of the agonist antibody for the taste/olfactory receptor. In other embodiments, an antagonist antibody can be used to bind any of the agonist antibodies described herein and likewise deactivate or inhibit the binding affinity of the agonist antibody for a taste/olfactory receptor. In some embodiments, the antagonist ligand, antagonist antibody, or antigen-binding fragment thereof, specifically binds the Fc fragment of the agonist antibody or antigen-binding fragment, thereby deactivating or inhibiting the ability of the agonist antibody to bind and activate the taste/olfactory receptor.

The edible compositions described herein, containing an antagonist ligand, antagonist antibody or fragment thereof, can be formulated as a seasoning that can be added to existing foods. Alternatively, the edible compositions described herein, containing an antagonist ligand, antagonist antibody or fragment thereof, can be incorporated into foods during processing.

According to one exemplary embodiment, the present technology provides an edible composition including an isolated “antagonist ligand” or an isolated “antagonist antibody” or an antigen-binding fragment thereof, which specifically binds a taste receptor ligand. The term “antagonist” is used in the broadest sense, and includes any molecule that partially or fully blocks or inhibits, one or more taste receptors or olfactory receptors disclosed herein, and thereby deactivates the receptor(s). In some embodiments, the isolated antagonist ligand, isolated antagonist antibody or antigen-binding fragment thereof prevents activation of the taste receptor upon binding the taste receptor ligand. In some embodiments, the taste receptor ligand is a tastant. In some embodiments, the tastant is a tastant is selected from the group consisting of a sweet substance, sour substance, bitter substance, and a savory substance.

In some embodiments, the edible composition further includes an isolated “agonist antibody” or an antigen-binding fragment thereof, which specifically binds and activates one or more taste receptors or specifically binds a taste receptor ligand that is an antagonist for the taste receptor. The term “agonist” is used in the broadest sense and includes any molecule that mimics the biological activity of a native ligand of one or more taste receptors or olfactory receptors disclosed herein, and thereby activates the receptor(s). In some embodiments, the isolated agonist antibody or antigen-binding fragment activates the taste receptor upon binding. In some embodiments, the taste receptor ligand is an agonist for the taste receptor. In some embodiments, the taste receptor ligand that is an agonist for the taste receptor is the isolated agonist antibody or the antigen-binding fragment of the composition. In some embodiments, the taste receptor ligand is an antagonist for the taste receptor.

In some embodiments, at least one of the antibodies or antigen-binding fragments is in a humanized form; is in chimeric form; is, or is from, a monoclonal antibody; is, or is from, a single-chain antibody; is, or is comprised in, a bispecific antibody; is, or is comprised in, a multispecific antibody. In some embodiments, at least one of the antibodies or antigen-binding fragments is or is from, a monoclonal antibody. In some embodiments, at least one of the antibodies or antigen-binding fragments is, or is comprised in, a multispecific antibody. In some embodiments, at least one of the antibodies or antigen-binding fragments is produced by a hybridoma cell line. In some embodiments, the edible composition includes an antigen-binding fragment selected from an Fab fragment, an F(ab′)2 fragment, or an Fv fragment. In some embodiments, the antagonist ligand, antagonist antibody or antigen-binding fragment thereof, specifically binds the Fc fragment of the isolated agonist antibody or the antigen-binding fragment thereof, which specifically binds the taste receptor.

In some embodiments, the antagonist antibody or antigen-binding fragment thereof is, or is comprised in, a bispecific antibody. In some embodiments, the bispecific antagonist antibody or antigen-binding fragment thereof includes an allosteric binding site. In some embodiments, the agonist antibody or antigen-binding fragment thereof is, or is comprised in, a bispecific antibody. In some embodiments, the bispecific agonist antibody or antigen-binding fragment thereof includes an allosteric binding site. In some embodiments, the taste receptor is a heterodimer, and wherein the bispecific agonist antibody or antigen-binding fragment thereof specifically binds the same polypeptide subunit of the heterodimer. In some embodiments, the taste receptor is a heterodimer, and wherein the bispecific agonist antibody or antigen-binding fragment thereof specifically binds different polypeptide subunits of the heterodimer.

According to another exemplary embodiment, the present technology provides an edible composition including an isolated bispecific “agonist antibody” or a bispecific antigen-binding fragment thereof, which specifically binds more than one epitope on a taste receptor and activates the taste receptor upon binding.

In some embodiments, the bispecific agonist antibody or antigen-binding fragment thereof includes an allosteric binding site. In some embodiments, the taste receptor is a heterodimer, and wherein the bispecific agonist antibody or antigen-binding fragment thereof specifically binds the same polypeptide subunit of the heterodimer. In some embodiments, the taste receptor is a heterodimer, and wherein the bispecific agonist antibody or antigen-binding fragment thereof specifically binds different polypeptide subunits of the heterodimer.

According to an additional exemplary embodiment, the present technology provides an edible/inhalable composition including at least one agonist antibody, described herein, and at least one antagonist ligand or antagonist antibody described herein. In some embodiments, the composition modulates the activity of one or more taste/olfactory receptors.

### Taste Receptors

The sense of taste is mediated by taste receptor cells that are bundled in clusters called taste buds. Taste receptor cells sample oral concentrations of a large number of small molecules and/or proteins and report a sensation of taste to centers in the brainstem. In most animals, including humans, taste buds are most prevalent on small pegs of epithelium on the tongue called papillae.

A large number of molecules elicit taste sensations through a relatively small number of taste receptors. Further, individual taste receptor cells apparently bear receptors for one type of taste. In other words, within a taste bud, some taste receptor cells sense sweet, while others have receptors for bitter, sour, salty and umami (i.e., savory) tastes.

As noted, in one embodiment, the present technology provides an edible composition including at least one agonist antibody, described herein, and/or at least one antagonist ligand or antagonist antibody described herein. The agonist antibody can activate a taste receptor, whereas the antagonist ligand or antagonist antibody can prevent activation of the taste receptor.

In some embodiments, the taste receptor is a human taste receptor. In some embodiments, the taste receptor is a non-human taste receptor. In some embodiments, the taste receptor is a heterodimer including two polypeptide subunits.

The pleasant tastes, sweet and umami (i.e., savory), are mediated by a family of three “TAS1” receptor subunits, TAS1R1 (SEQ ID NO:1), TAS1R2 (SEQ ID NO:2), and TAS1R3 (SEQ ID NO:3) that assemble in pairs (i.e., dimers). Diverse molecules that lead to a sensation of sweet bind to a receptor formed from TAS1R2 and TAS1R3 subunits. The receptor formed as a complex of TAS1R1 and TAS1R3 binds L-glutamate and L-amino acids, resulting the umami taste.

In some embodiments, the taste receptor is a sweet receptor. In some embodiments, the taste receptor is a heterodimer including two polypeptide subunits independently selected from the Type 1 (TAS1 “sweet”) family of polypeptide subunits. In some embodiments, the sweet taste receptor includes TAS1R2 and TAS1R3 polypeptide subunits.

In some embodiments, each polypeptide subunit from the TAS1 family is independently selected from the group consisting of TAS1R1, TAS1R2, and TAS1R3.

In some embodiments, the taste receptor is an umami receptor. In some embodiments, the umami receptor includes TAS1R1 and TAS1R3 polypeptide subunits.

Bitter taste results from binding of diverse molecules to a family of numerous “TAS2” receptors. In some embodiments, the taste receptor is a heterodimer including two polypeptide subunits independently selected from the Type 2 (TAS2 “bitter”) family of polypeptide subunits.

In certain embodiments, each polypeptide subunit from the TAS2 family is independently selected from the group consisting of TAS2R1 (SEQ ID NO:4), TAS2R3 (SEQ ID NO:5), TAS2R4 (SEQ ID NO:6), TAS2R5 (SEQ ID NO:7), TAS2R7 (SEQ ID NO:8), TAS2R8 (SEQ ID NO:9), TAS2R9(SEQ ID NO:10), TAS2R10 (SEQ ID NO:11), TAS2R13 (SEQ ID NO:12), TAS2R14 (SEQ ID NO:13), TAS2R16 (SEQ ID NO:14), TAS2R19 (SEQ ID NO:15), TAS2R20 (SEQ ID NO:16), TAS2R30 (SEQ ID NO:17), TAS2R31 (SEQ ID NO:18), TAS2R38 (SEQ ID NO:19), TAS2R39 (SEQ ID NO:20), TAS2R40 (SEQ ID NO:21), TAS2R41 (SEQ ID NO:22), TAS2R42 (SEQ ID NO:23), TAS2R43 (SEQ ID NO:24), TAS2R44 (SEQ ID NO:25), TAS2R45 (SEQ ID NO:26), TAS2R46 (SEQ ID NO:27), TAS2R49 (SEQ ID NO:28), TAS2R50 (SEQ ID NO:29), and TAS2R60 (SEQ ID NO:30). In some embodiments, the taste receptor includes the TAS2R38 (SEQ ID NO:19) polypeptide subunit.

In some embodiments, the taste receptor is a sour receptor. Sour taste is generally mediated by ion channels. Proposed receptors for sour taste include the hyperpolarization-activated cyclic nucleotide-gated (HCN) channels, HCN1 (SEQ ID NO:31) and HCN4 (SEQ ID NO:32), the amiloride-sensitive cation channel 1 ACCN1 (SEQ ID NO:33), and the two-pore domain potassium channel TASK-1 (SEQ ID NO:34). In some embodiments, the taste receptor is a sour receptor. In some embodiments, the sour receptor is selected from the group consisting of a hyperpolarization-activated cyclic nucleotide-gated channel (HCN1 and HCN4), the amiloride-sensitive cation channel 1 (ACCN1), and the two-pore domain potassium channel (TASK-1).

In some embodiments, the taste receptor is a salty receptor. The molecular basis for salty taste reception is poorly characterized relative to the basis for other tastes. The epithelial sodium channel (ENaC) receptor has been proposed to contribute to salt taste reception. In some embodiments, the taste receptor is a salt taste receptor. In some embodiments, the salt taste receptor is the epithelial sodium channel (ENaC) receptor.

Representative taste receptors and their subunit polypeptides are shown below in Table 1.

TABLE 1
Human Taste Cell Receptors and Subunit Polypeptides

| Human | | | | |
|---|---|---|---|---|
| Taste | | Fig. | NCBI Accession | |
| Receptor | SEQ ID | # | #/Gene ID | Reference |
| TAS1R1 | SEQ ID | 1 | AAI36516.1/ | Strausberg, R. L., et al., Proc. Natl. |
| | NO: 1 | | 80835 | Acad. Sci. U.S.A. 99 (26), |
| | | | | 16899-16903 (2002) |
| TAS1R2 | SEQ ID | 2 | NP_689418 | Kyriazis, G. A., et al., Proc. Natl. |
| | NO: 2 | | XP 944007/ | Acad. Sci. U.S.A. 109 (8), E524- |
| | | | 80834 | E532 (2012) |
| TAS1R3 | SEQ ID | 3 | NP_689414.1/ | Maitrepierre, E., et al., Protein |
| | NO: 3 | | 83756 | Expr. Purif. 83 (1), 75-83 (2012) |
| TAS2R1 | SEQ ID | 4 | EAX08075.1/ | Venter J. C., et al., Science 291 |
| | NO: 4 | | 50834 | (5507), 1304-1351 (2001) |
| TAS2R3 | SEQ ID | 5 | EAW83982.1/ | Venter J. C., et al., supra. |
| | NO: 5 | | 50831 | |
| TAS2R4 | SEQ ID | 6 | EAW83983.1/ | Venter J. C., et al., supra. |
| | NO: 6 | | 50832 | |
| TAS2R5 | SEQ ID | 7 | EAW83984.1/ | Venter J. C., et al., supra. |
| | NO: 7 | | 54429 | |
| TAS2R7 | SEQ ID | 8 | EAW96208.1/ | Venter J. C., et al., supra. |
| | NO: 8 | | 50837 | |
| TAS2R8 | SEQ ID | 9 | EAW96209.1/ | Venter J. C., et al., supra. |
| | NO: 9 | | 50836 | |
| TAS2R9 | SEQ ID | 10 | EAW96210.1/ | Venter J. C., et al., supra. |
| | NO: 10 | | 50835 | |
| TAS2R10 | SEQ ID | 11 | EAW96211.1/ | Venter J. C., et al., supra. |
| | NO: 11 | | 50839 | |
| TAS2R13 | SEQ ID | 12 | EAW96218.1/ | Venter J. C., et al., supra. |
| | NO: 12 | | 50838 | |
| TAS2R14 | SEQ ID | 13 | NP_076411.1/ | Campa, D., et al., BMC Med. |
| | NO: 13 | | 50840 | Genet. 11, 88 (2010) |
| TAS2R16 | SEQ ID | 14 | NP_058641.1/ | Wang, J. C., et al., Alcohol. Clin. |
| | NO: 14 | | 50833 | Exp. Res. 31 (2), 209-215 (2007) |
| TAS2R19 | SEQ ID | 15 | NP_795369.1/ | Reed, D. R., et al., Hum. Mol. |
| | NO: 15 | | 259294 | Genet. 19 (21), 4278-4285 |
| | | | | (2010) |
| TAS2R20 | SEQ ID | 16 | NP_795370.2/ | Zhang, Y., et al., Cell 112 (3), |
| | NO: 16 | | 259295 | 293-301 (2003) |
| TAS2R30 | SEQ ID | 17 | NP_001091112 | Go, Y., et al., Genetics 170 (1), |
| | NO: 17 | | XP_001129113/ | 313-326 (2005) |
| | | | 259293 | |
| TAS2R31 | SEQ ID | 18 | NP_795366.2/ | Roudnitzky, N., et al., Hum. Mol. |
| | NO: 18 | | 259290 | Genet. 20 (17), 3437-3449 (2011) |
| TAS2R38 | SEQ ID | 19 | AFH77562.1/ | Wooding, S. P. |
| | NO: 19 | | 5726 | Direct Submission Dec. 10, 2011 |
| TAS2R39 | SEQ ID | 20 | AAI14953.1/ | Strausberg, R. L., et al., supra |
| | NO: 20 | | 259285 | |
| TAS2R40 | SEQ ID | 21 | EAL23782.1/ | Scherer, S. W., et al., Science 300 |
| | NO: 21 | | 259286 | (5620), 767-772 (2003) |
| TAS2R41 | SEQ ID | 22 | AAI01159.1/ | Strausberg, R. L., et al., Proc. |
| | NO: 22 | | 259287 | Natl. Acad. Sci. U.S.A. 99 (26), |
| | | | | 16899-16903 (2002) |
| TAS2R42 | SEQ ID | 23 | NP_852094.1/ | Scherer, S. W., et al., supra |
| | NO: 23 | | 353164 | |
| TAS2R43 | SEQ ID | 24 | NP_795365.2/ | Hirai, R., et al., Ann. Otol. |
| | NO: 24 | | 259289 | Rhinol. Laryngol. 121 (2), 113- |
| | | | | 118 (2012) |
| TAS2R44 | SEQ ID | 25 | EAW96226.1/ | Venter J. C., et al., supra. |
| | NO: 25 | | 259290 | |
| TAS2R45 | SEQ ID | 26 | EAW96228.1/ | Venter J. C., et al., supra. |
| | NO: 26 | | 259291 | |
| TAS2R46 | SEQ ID | 27 | NP_795368.2/ | Zhang, Y., et al., supra |
| | NO: 27 | | 259292 | |
| TAS2R49 | SEQ ID | 28 | EAW96224.1/ | Venter J. C., et al., supra. |
| | NO: 28 | | 259295 | |
| TAS2R50 | SEQ ID | 29 | NP_795371.2/ | Akao, H., et al., Atherosclerosis |
| | NO: 29 | | 259296 | 220 (2), 456-462 (2012) |
| TAS2R60 | SEQ ID | 30 | NP_803186.1/ | Go, Y., et al., supra |
| | NO: 30 | | 338398 | |
| HCN1 | SEQ ID | 31 | NP_066550.2/ | Klueva, J., et al., Neurosignals 20 |
| | NO: 31 | | 348980 | (1), 35-47 (2012) |
| HCN4 | SEQ ID | 32 | NP_005468.1/ | Ellinor, P. T., et al., Nat. Genet. |
| | NO: 32 | | 10021 | 44 (6), 670-675 (2012) |
| ACCN1 | SEQ ID | 33 | AAH75043.1/ | Strausberg, R. L., et al., supra |
| | NO: 33 | | 40 | |
| TASK-1 | SEQ ID | 34 | O14649.1/ | Duprat, F., et al., EMBO J. 16 |
| | NO: 34 | | 3777 | (17), 5464-5471 (1997) |

As noted, an agonist includes any molecule that mimics a biological activity of a native ligand of a taste or olfactory receptor disclosed herein, and thereby activates the receptor. The present technology provides, in one embodiment, an edible composition including an isolated “antagonist ligand” or an isolated “antagonist antibody” or an antigen-binding fragment thereof, which specifically binds the agonist antibody or antigen-binding fragment, or one or more agonist ligands that activate a taste receptor.

Such agonist ligands that activate a taste receptor are selected from the group consisting of a sweet substance, sour substance, bitter substance, savory substance, and salty substance (e.g., a small molecule, salt, or polypeptide).

In some embodiments, the agonist ligand is a sweet substance selected from the group consisting of glucose, fructose, galactose, sucrose, lactose, stevia, aspartame, sucralose, neotame, acesulfame potassium, and saccharin.

In some embodiments, the agonist ligand is a sour substance selected from the group consisting of dilute hydrochloric acid, tartaric acid, citric acid, and carbonic acid.

In some embodiments, the agonist ligand is a bitter substance selected from the group consisting of acetylthiourea, cycloheximide, denatonium benzoate (N-benzyl-2-(2,6-dimethylphenylamino)-N,N-diethyl-2-oxoethanaminium benzoate), N,N-dimethylthioformamide, N,N′-diphenylthiourea, N,N′-ethylenethiourea, N-ethylthiourea, a β-glucopyranoside, methimazol, 4(6)-methyl-2-thiouracil, N-methylthiourea, phenylthiocarbamide (PTC), 6-phenyl-2-thiouracil, 5-propyl-2-thiouracil, 6-propyl-2thiouracil (PROP), tetramethyl thiourea, thioacetamide, thioacetanilid, and 2-thiobarbituric acid.

In some embodiments, the agonist ligand is a savory substance such as monosodium glutamate (MSG).

In some embodiments, the agonist ligand is a salty substance such as sodium chloride.

### Foods and Beverages

As noted above, the edible compositions described herein, containing an agonist antibody or fragment thereof, can formulated as a seasoning that can be added to existing foods. Alternatively, the edible compositions described herein containing an agonist antibody or fragment thereof, can be incorporated into foods during processing to provide flavor to otherwise bland foods.

Further, the edible compositions described herein, containing an antagonist ligand, antagonist antibody or fragment thereof, can likewise be formulated as a seasoning that can be added to existing foods. Alternatively, the edible compositions described herein, containing an antagonist ligand, antagonist antibody or fragment thereof, can also be incorporated into foods during processing.

The edible compositions described herein may also include one or more dietary additives such a protein, carbohydrate, lipid, vitamin, mineral, sweetening agent, food flavoring agent or enhancer, food color, antimicrobial agent, antioxidant, surface modifying agent, emulsification agent, or a combination thereof.

The edible compositions described herein may include edible dietary proteins that derive from any source of edible protein, such as animal, vegetable, dairy products, or a combination thereof, including soy, whey, or tofu. Examples of suitable vegetable protein sources are soybeans, safflower seed, corn, peanuts, wheat, peas, sunflower seed, cottonseed, coconut, rapeseed, sesame seed, leaf proteins, single cell proteins such as yeast, and the like.

In some embodiments, the edible compositions described herein include soy protein or any soy-based additive such as soy flours and grits, soy protein concentrates and soy protein isolates. Additionally, the edible compositions described herein may include any tofu-based additive made by coagulating soybean milk and pressing the resulting curds.

Additionally, the edible compositions described herein may include protein from animal protein sources, such as those derived from milk, poultry, meat, and/or fish. A typical example of a suitable animal protein is egg albumin. Additionally, the edible compositions described herein may include any whey-based additive derived from the processing of whey, the liquid material created as a by-product of cheese production. In some embodiments, the edible compositions described herein include a mixture of globular proteins isolated from whey.

The edible compositions described herein may include an amount of edible dietary protein that can and will vary. For example, the edible compositions described herein may include about 1%, 5%, 10%, 20%, 30%, 40%, 50%, 60%, 70%, 80%, 90%, 99%, or a range between and including any two of these values, by weight of edible protein.

As noted, in some embodiments, the edible compositions described herein include an edible dietary carbohydrate. Representative carbohydrates include sugars (glucose, fructose, galactose, sucrose, lactose, etc.) starches, and the like.

The edible compositions described herein may include an amount of edible dietary carbohydrates that can and will vary. For example, the edible compositions described herein may include about 1%, 5%, 10%, 20%, 30%, 40%, 50%, 60%, 70%, 80%, 90%, 99%, or a range between and including any two of these values, by weight of carbohydrates.

As noted, in some embodiments, the edible compositions described herein include edible dietary fats and lipids. Examples of edible dietary fats and lipids include, but are not limited to, saturated and unsaturated fatty acids and/or glycerides derived from animal, vegetable, or marine fats and oils, including synthetically prepared shortenings. These glycerides can contain saturated or unsaturated “long chain” acyl radicals having from about 12 to about 22 carbon atoms such as lauroyl, lauroleoyl, myristoyl, myristoleoyl, palmitoyl, palmitoleyl, stearoyl, oleoyl, linoleoyl, linolenoyl, arachidoyl, arachidonyl, behenoyl, erucoyl, and the like, and are generally obtained from edible fats and oils such as cottonseed oil, soybean oil, coconut oil, rapeseed oil, peanut oil, olive oil, palm oil, palm kernel oil, sunflower seed oil, rice bran oil, corn oil, sesame seed oil, safflower oil, wallflower oil, nasturtium seed oil, sardine oil, herring oil, menhaden oil, pilchard oil, lard, tallow and the like.

The type of fat that is added to the edible compositions described herein can be selected to help mimic the texture and flavor of any particular food. For instance, a vegetable fat such as cottonseed oil may be incorporated into the edible compositions described herein when an unsaturated fat is desired to prepare foods containing no animal products. Alternatively, animal fats may be incorporated into the edible compositions described herein to achieve fat levels that replicate those found in various meats. Other ingredients such as flavoring agents, coloring, seasoning, and the like can also be added to the edible compositions described herein to simulate any particular food.

The edible compositions described herein may include an amount of fats and lipids that can and will vary. For example, the edible compositions described herein may include about 1%, 5%, 10%, 20%, 30%, 40%, 50%, 60%, 70%, 80%, 90%, 99%, or a range between and including any two of these values, by weight of fats and lipids.

In some embodiments, the edible compositions described herein further include one or more vitamins and/or minerals. Representative vitamins include, but are not limited to, the group of fat soluble vitamins consisting of retinol (vitamin A), calciferol (vitamin D), tocopherol (vitamin E), phytomenadione (vitamin K1), water soluble vitamins group consisting of thiamine (vitamin B1), riboflavin (vitamin B2), pyridoxine (vitamin B6), nicotinamide (niacin), pantothenic acid, biotin, folic acid, cyanocobalamin (vitamin B12), ascorbic acid (vitamin C), polyunsaturated fatty acids (PUFA), and the like.

Representative minerals include, but are not limited to, salts of sodium, potassium, magnesium, calcium, chloride, phosphate, iron, copper, zinc, manganese, cobalt, vanadium, chromium, selenium, molybdenum, nickel, boron, silica, silicon, fluorine, iodine, arsenic and the like.

The edible compositions described herein may include an amount of one or more vitamins and/or minerals that can and will vary. For example, the edible compositions described herein may include about 1%, 5%, 10%, 20%, 30%, 40%, 50%, or a range between and including any two of these values, by weight of one or more vitamins and/or minerals.

In some embodiments, the edible compositions described herein may include, without being limited to, a sweetening agent, a food flavoring agent or enhancer, a food color, an antimicrobial agent, an antioxidant, a surface modifying agent, an emulsification agent or a mixture thereof.

Examples of sweetening agents include, natural sweeteners, such as a sugar, and artificial sweetening products such as saccharin, cyclamate, monellin, thaumatins, curculin, miraculin, stevioside, phyllodulcin, glycyrrhizin, nitroanilines, dihydrochalcones, dulcin, suosan, guanidines, oximes, oxathiazinone dioxides, aspartame, alitame, and the like. Natural sweeteners can also be monosaccharides and oligosaccharides. Examples of monosaccharides include, but are not limited to, galactose, fructose, glucose, sorbose, agatose, tagatose and xylose. Examples of oligosaccharides include sucrose, lactose, lactulose, maltose, isomaltose, maltulose, saccharose and trehalose. Other sweetening agents that can also be used include, but are not limited to, high fructose corn syrup or sugar alcohols.

Examples of food flavoring agents or enhancers include, but are not limited to, monosodium glutamate, maltol, 5′-mononucleotides, such as inosine, and the like.

Examples of food colors include, but are not limited to, tartrazine, riboflavin, curcumin, zeaxanthin, (3-carotene, bixin, lycopene, canthaxanthin, astaxanthin, |3-apo-8′ carotenal, carmoisine, amaranth, Ponceau 4R (E124), Carmine (E120), anthocyanidin, erythrosine, Red 2G, Indigo Carmine (E 132), Patent Blue V (E 131), Brilliant blue, chlorophyll, chlorophyllin copper complex, Green S (E142), Black BN (E151), and the like.

Examples of antimicrobial agents include, but are not limited to, benzoic acid, PHB esters, sorbic acid, propionic acid, acetic acid, sodium sulfite and sodium metabisulfite, diethyl pyrocarbonate, ethylene oxide, propylene oxide, nitrite, nitrate, antibiotics, diphenyl, o-phenylphenol, thiabendazole and the like.

Examples of antioxidant agents include, but are not limited to, tocopherols, 2,6-di-tert-butyl-p-cresol (BHT), tert-butyl-4-hydroxyanisole (BHA), propylgallate, octylgallate, dodecylgallate, ethoxyquin, ascorbyl palmitate, ascorbic acid and the like.

Examples of surface modifying agents include, but are not limited to, mono-, diaglycerides and derivatives, sugar esters, sorbitan fatty acid esters, polyoxyethylene sorbitan esters, stearyl-2-lactylate and the like.

In some embodiments, the edible compositions described herein can be added to a conventional food such as meat, ground meat, seafood, poultry, a cereal food product, a baked good product, a health food product, dairy product, fruit, vegetable, bakery item, confection, pet food product, or animal feed.

Meat and ground meat refer to animal flesh that is eaten as food. As used herein, meat includes the flesh of mammalian species (pigs, cattle, lambs, etc.) raised and prepared for human consumption, in addition to fish and other seafood, poultry, and other animals.

A cereal food product is a food product derived from the processing of a cereal grain. A cereal grain includes any plant from the grass family that yields an edible grain (seed). The most popular grains are barley, corn, millet, oats, quinoa, rice, rye, sorghum, triticale, wheat and wild rice. Examples of a cereal food product include, but are not limited to, whole grain, crushed grain, grits, flour, bran, germ, breakfast cereals, extruded foods, pastas, and the like.

A baked good product includes any of the cereal food products mentioned above and has been baked or processed in a manner comparable to baking, i.e., to dry or harden by subjecting to heat. Examples of a baked good product include, but are not limited to, bread crumbs, baked snacks, mini-biscuits, mini-crackers, mini-cookies, and mini-pretzels.

A health food product is any food product that imparts a health benefit. Many oilseed-derived food products may be considered as health foods. There can be mentioned soybeans, flax seed, sesame seed, pumpkin seeds, sunflower seeds, or food products processed from these seeds or which are incorporated into foods, such as soy nuggets and soy nuts. In addition health food products include oilseed-derived food products, fruit-derived food products, such as fruit bits, dried berries, and the like.

A pet food product is a product intended to be fed to a pet such as a dog, cat, bird, reptile, fish, rodent and the like. These products can include the cereal and health food products above, as well as meat and meat byproducts, grass and hay products, including but not limited to alfalfa, timothy, oat or brome grass and the like.

Animal feed is a product intended to be fed to animals such as turkeys, chickens, cattle, horses, swine and the like. As with the pet foods above, these products can include cereal, meat and meat byproducts, and grass and hay products.

In an additional aspect, a food or beverage is provided that includes any of the edible compositions described herein. In some embodiments, the food is a seasoning. In other embodiments, the food includes tofu, soy, or whey. In some embodiments, the beverage is an energy beverage, weight-loss beverage, and a beverage concentrate that is diluted with water by the consumer.

### Olfactory Receptors

Olfactory receptors are responsible for the detection of odor molecules. In vertebrates, olfactory receptors are located in the cilia of the olfactory sensory neurons. Activated olfactory receptors initiate a signal transduction cascade that ultimately produces a nerve impulse which is transmitted to the brain. These receptors are members of class A rhodopsin-like family of G protein-coupled receptors (GPCRs). The olfactory receptors are a multigene family consisting of over 900 genes in humans. See Malnic et al., Proc. Nat. Acad. Sci. USA Feb. 24, 2004, 10(8) 2584-2589.

The present technology also provides, in another exemplary embodiment, a composition for intranasal administration including an isolated “agonist antibody” or an antigen-binding fragment thereof, which specifically binds one or more olfactory receptors. In some embodiments, the isolated agonist antibody or antigen-binding fragment activates the olfactory receptor upon binding.

The present technology also provides, in another exemplary embodiment, a composition for intranasal administration including an isolated “antagonist ligand,” an isolated “antagonist antibody” or an antigen-binding fragment thereof, which specifically binds an olfactory receptor ligand. In some embodiments, the olfactory receptor ligand is an odorant.

In some embodiments, the composition for intranasal administration further includes an isolated “agonist antibody” or an antigen-binding fragment thereof, which specifically binds and activates one or more olfactory receptors. In some embodiments, the olfactory receptor ligand is an agonist for the olfactory receptor. In some embodiments, the olfactory receptor ligand that is an agonist for the olfactory receptor is the isolated agonist antibody or the antigen-binding fragment of the composition. In some embodiments, the olfactory receptor ligand is an antagonist for the olfactory receptor.

In some embodiments, at least one of the antibodies or antigen-binding fragments is in a humanized form; is in chimeric form; is, or is from, a monoclonal antibody; is, or is from, a single-chain antibody; is, or is comprised in, a bispecific antibody; is, or is comprised in, a multispecific antibody. In some embodiments, at least one of the antibodies or antigen-binding fragments is or is from, a monoclonal antibody. In some embodiments, at least one of the antibodies or antigen-binding fragments is, or is comprised in, a multispecific antibody. In some embodiments, at least one of the antibodies or antigen-binding fragments is produced by a hybridoma cell line. In some embodiments, the composition for intranasal administration includes an antigen-binding fragment selected from an Fab fragment, an F(ab′)2 fragment, or an Fv fragment. In some embodiments, the antagonist ligand, antagonist antibody or antigen-binding fragment thereof, specifically binds the Fc fragment of the isolated agonist antibody or the antigen-binding fragment thereof, which specifically binds the olfactory receptor.

In some embodiments, the antagonist antibody or antigen-binding fragment thereof is, or is comprised in, a bispecific antibody. In some embodiments, the bispecific antagonist antibody or antigen-binding fragment thereof includes an allosteric binding site. In some embodiments, the agonist antibody or antigen-binding fragment thereof is, or is comprised in, a bispecific antibody. In some embodiments, the bispecific agonist antibody or antigen-binding fragment thereof includes an allosteric binding site.

According to another exemplary embodiment, the present technology provides a composition for intranasal administration including at least one agonist antibody as described herein and at least one antagonist ligand or antagonist antibody as described herein. In some embodiments, the composition modulates the activity of one or more olfactory receptors.

In some embodiments, the one or more olfactory receptors are selected from the class A rhodopsin-like family of G protein-coupled receptors (GPCRs). In some embodiments, the composition is formulated as an aerosol, ointment, lotion, wash, or intranasal powder.

In some embodiments, the agonist ligand that activates one or more olfactory receptors is a compound with a molecular weight of less than 500 selected from the group consisting of an alcohol, ester, aldehyde, ketone, lactone, thiol, and amine.

### Compositions for Intranasal Administration

Techniques for producing intranasal formulations of antibodies are known in the art and are described, for instance, in U.S. Pat. Nos. 7,323,183, 8,226,982, published U.S. Patent Applications 2006/0188496, 2011/0229461, and references cited therein.

According to one embodiment, a composition for intranasal administrations described herein can be applied to the nose to contact the olfactory epithelium, particularly the olfactory cilia. Application of composition for intranasal administrations described herein leads to modulation of the sense of smell.

Suitable intranasal preparations include intranasal powders, metering aerosols containing propellant gases or intranasal solutions free from propellant gases. Intranasal powders according to the disclosure containing the active substance may consist solely of the abovementioned active substances or of a mixture of the abovementioned active substances with physiologically acceptable excipient. Other modes of administration are also possible, such as an ointment, lotion, and wash.

One exemplary formulation for intranasal delivery of the composition for intranasal administrations described herein is a liquid preparation, preferably an aqueous based preparation, suitable for application as drops into the intranasal cavity. For example, intranasal drops can be instilled in the intranasal cavity by tilting the head back sufficiently and apply the drops into the nares. The drops may also be snorted up the nose.

Topical administration of the composition for intranasal administrations described herein to the nose may also be by administration of a liquid solution or suspension formulation, for example employing a device such as a nebulizer, for example, a nebulizer connected to a compressor (e.g., the Pari LC-Jet Plus® nebulizer connected to a Pari Master® compressor manufactured by Pari Respiratory Equipment, Inc., Richmond, Va.).

The composition for intranasal administrations described herein can be delivered to the nose dispersed in a solvent, e.g., in the form of a solution or a suspension. It can be suspended in an appropriate physiological solution, e.g., saline or other pharmacologically acceptable solvent or a buffered solution. Buffered solutions known in the art may contain 0.05 mg to 0.15 mg disodium edetate, 8.0 mg to 9.0 mg NaCl, 0.15 mg to 0.25 mg polysorbate, 0.25 mg to 0.30 mg anhydrous citric acid, and 0.45 mg to 0.55 mg sodium citrate per 1 ml of water so as to achieve a pH of about 4.0 to 5.0. A suspension can employ, for example, freeze-dried (i.e., lyophilized) antibody.

In one embodiment the formulation is provided as a formulation for inhalation. Accordingly, a liquid preparation may be placed into an appropriate device so that it may be aerosolized for inhalation through the intranasal cavity. For example, the therapeutic agent may be placed into a plastic bottle atomizer. In one embodiment, the atomizer is advantageously configured to allow a substantial amount of the spray to be directed to the upper one-third region or portion of the intranasal cavity. Alternatively, the spray is administered from the atomizer in such a way as to allow a substantial amount of the spray to be directed to the upper one-third region or portion of the intranasal cavity. By “substantial amount of the spray” it is meant herein that at least about 50%, further at least about 70%, but preferably at least about 80% or more of the spray is directed to the upper one-third portion of the intranasal cavity.

Additionally, the liquid preparation may be aerosolized and applied via an inhaler, such as a metered-dose inhaler. One example of an exemplary device is that disclosed by Djupesland et al., U.S. Pat. No. 6,715,485, that includes a bi-directional delivery concept. In using the device, the end of the device having a sealing nozzle is inserted into one nostril and the patient or subject blows into the mouthpiece. During exhalation, the soft palate closes due to positive pressure thereby separating the intranasal and oral cavities. The combination of closed soft palate and sealed nozzle creates an airflow in which antibody particles are released entering one nostril, turning 180 degrees through the communication pathway and exiting through the other nostril, thus achieving bi-directional flow.

The propellant gases which can be used to prepare the intranasal aerosols are known in the art. Suitable propellant gases are selected from among hydrocarbons such as n-propane, n-butane or isobutane and halohydrocarbons such as chlorinated and/or fluorinated derivatives of methane, ethane, propane, butane, cyclopropane or cyclobutane. The abovementioned propellant gases may be used on their own or in mixtures thereof.

Particularly suitable propellant gases are halogenated alkane derivatives selected from among TG 11, TG 12, TG 134a and TG227. Of the abovementioned halogenated hydrocarbons, TG134a (1,1,1,2-tetrafluoroethane) and TG227 (1,1,1,2,3,3,3-heptafluoropropane) and mixtures thereof are particularly suitable.

The propellant-gas-containing intranasal aerosols may also contain other ingredients such as cosolvents, stabilisers, surface-active agents (surfactants), antioxidants, lubricants and means for adjusting the pH. All these ingredients are known in the art.

The propellant-gas-containing intranasal aerosols may contain up to 5% by weight of active antibody. The aerosols, for example, 0.002 to 5% by weight, 0.01 to 3% by weight, 0.015 to 2% by weight, 0.1 to 2% by weight, 0.5 to 2% by weight or 0.5 to 1% by weight of antibody ingredient.

The antibody can also be delivered in the form of a dry powder, as in known in the art. An example of a suitable device is the dry powder intranasal delivery device marketed under the name DirectHaler® intranasal, and which is disclosed in the international PCT publication No. WO96/222802. This device also enables closing of the passage between the intranasal and oral cavity during dose delivery.

These intranasal powders may include monosaccharides (e.g., glucose or arabinose), disaccharides (e.g., lactose, saccharose, maltose), oligo- and polysaccharides (e.g., dextrans), polyalcohols (e.g., sorbitol, mannitol, xylitol), salts (e.g., sodium chloride, calcium carbonate) or mixtures of these with one another. Mono- or disaccharides are suitably used, the use of lactose or glucose, particularly but not exclusively in the form of their hydrates.

### Antibodies

The antibody (i.e., immunoglobulin) or antibody fragment of the compositions described herein include antibodies or fragments that specifically bind to an epitope on a taste receptor or on an olfactory receptor. The antibodies or antibody fragments of the compositions described herein further include those that specifically bind to an agonist (e.g., sucrose) that itself activates a taste receptor or an olfactory receptor. The antibody or antibody fragment may be a polyclonal or a monoclonal antibody. Antibodies may be obtained by injecting a desired antigen into a subject, typically an animal such as a mouse, as well established in the art. The antigen may be a full-length polypeptide, or fragment thereof, from a taste receptor, or a subunit thereof. The antigen may also be a full-length polypeptide, or fragment thereof, from an olfactory receptor, or a subunit thereof. The antigen may further be an agonist molecule (e.g., sucrose) that activates a taste receptor or an olfactory receptor. The antigen, along with an adjuvant such as Freund's complete adjuvant, may be injected into the subject multiple times subcutaneously or intraperitoneally.

The antibody or antibody fragment of the compositions described herein include commercially available antibodies and their corresponding blocking peptides. Non-limiting suppliers of antibodies include Santa Cruz Biotechnology, Inc. (Santa Cruz, Calif.) and MBL International, Inc., (Woburn, Mass.). Representative commercially available antibodies or blocking peptides, such as those available from Santa Cruz Biotechnology, are provided in Table 2 below. The antibodies may be used as agonist (e.g., primary) antibodies that specifically bind taste/olfactory receptors, whereas the blocking peptides may be used as antagonist (e.g., “secondary”) antibodies specifically bind the primary antibodies rather than a taste/olfactory receptors.

TABLE 2
Representative Commercially Available Antibodies and Blocking Peptides

| | Santa Cruz | | | Blocking |
|---|---|---|---|---|
| | Biotechnology | | Epitope within the | peptide |
| Antibody | Catalog # | Host/Isotype | human receptor | Catalog # |
| TAS1R1 | (H-120) sc-50307 | rabbit | amino acids 274-393 | |
| | | polyclonal IgG | mapping within an | |
| | | | extracellular domain | |
| TAS1R2 | (V-20) sc-22453 | goat | within an N-terminal | sc-22453 P |
| | | polyclonal IgG | extracellular domain | |
| TAS1R3 | (V-20) sc-34058 | goat | within an N-terminal | sc-34058 P |
| | | polyclonal IgG | extracellular domain | |
| TAS2R1 | (H-180) sc-67106 | rabbit | amino acids 1-180 | |
| | | polyclonal IgG | mapping at the N- | |
| | | | terminus | |
| TAS2R3 | (E-13): sc-163425 | goat | within a cytoplasmic | sc-163425 P |
| | | polyclonal IgG | domain | |
| TAS2R4 | (T-13): sc-169494 | goat | within a cytoplasmic | sc-169494 P |
| | | polyclonal IgG | domain | |
| TAS2R7 | (H-60): sc-67111 | rabbit | amino acids 141-200 | |
| | | polyclonal IgG | mapping within an | |
| | | | internal region | |
| TAS2R10 | (D-12): sc-169473 | goat | within an extracellular | sc-169473 P |
| | | polyclonal IgG | domain | |
| TAS2R16 | (K-14): sc-163420 | goat | within a cytoplasmic | sc-163420 P |
| | | polyclonal IgG | domain | |
| TAS2R38 | (H-220): sc-67108 | rabbit | amino acids 1-220 | |
| | | polyclonal IgG | mapping at the N- | |
| | | | terminus | |
| TAS2R40 | (G-15): sc-165637 | goat | within an N-terminal | sc-165637 P |
| | | polyclonal IgG | cytoplasmic domain | |
| TAS2R43 | (N-12): sc-34851 | goat | within an N-terminal | sc-34851 P |
| | | polyclonal IgG | cytoplasmic domain | |
| TAS2R44 | (A-12): sc-34728 | goat | within an N-terminal | sc-34728 P |
| | | polyclonal IgG | cytoplasmic domain | |
| TAS2R45 | (Q-12): sc-34855 | goat | within an extracellular | sc-34855 P |
| | | polyclonal IgG | domain | |
| TAS2R46 | (S-12): sc-34734 | goat | within a C-terminal | sc-34734 P |
| | | polyclonal IgG | extracellular domain | |
| TAS2R47 | (L-12): sc-34859 | goat | within a C-terminal | sc-34859 P |
| | | polyclonal IgG | extracellular domain | |
| TAS2R49 | (S-12): sc-34531 | goat | within an N-terminal | sc-34531 P |
| | | polyclonal IgG | extracellular domain | |
| TAS2R50 | (S-12): sc-34535 | goat | within a C-terminal | sc-34535 P |
| | | polyclonal IgG | extracellular domain | |
| TAS2R60 | (C-11) sc-169502 | goat | within a C-terminal | sc-169502 P |
| | | polyclonal IgG | cytoplasmic domain | |
| | | | (h) | |
| HCN1 | (V-17) sc-19706 | rabbit | C-terminus (h) | sc-19706 P |
| | | polyclonal IgG | | |
| HCN4 | (H-300) sc-28750 | rabbit | 904-1203 (h) | |
| | | polyclonal IgG | | |
| ACCN1 | (E-20) sc-22333 | goat | N-terminus (h) | sc-22333 P |
| (ASIC2) | | polyclonal IgG | | |
| TASK-1 | (H-50) sc-28635 | rabbit | 295-344 (h) | |
| | | polyclonal IgG | | |

In some embodiments, at least one of the antibodies or antigen-binding fragments described herein is in a humanized form; is in chimeric form; is, or is from, a monoclonal antibody; is, or is from, a single-chain antibody; is, or is included in, a bispecific antibody; is, or is included in, a multispecific antibody.

In some embodiments, at least one of the antibodies or antigen-binding fragments described herein is or is from, a monoclonal antibody. In some embodiments, at least one of the antibodies or antigen-binding fragments described herein is, or is included in, a bispecific antibody. In some embodiments, at least one of the antibodies or antigen-binding fragments described herein is, or is included in, a multispecific antibody.

In some embodiments, at least one of the antibodies or antigen-binding fragments described herein is produced by a hybridoma cell line. In some embodiments, at least one of the antibodies or antigen-binding fragments described herein selected from an Fab fragment, an F(ab′)2 fragment, or an Fv fragment.

As used herein, the terms “antibody” include glycoproteins including at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region (C_{H}). The heavy chain constant region is comprised of three domains, CH1, CH2 and CH3. Each light chain is comprised of a light chain variable region (abbreviated herein as LCVR or V_{L}) and a light chain constant region (C_{L}). The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged from amino-terminus to carboxy-terminus in the following order: FR1, CDR1, FR2, CDR2, FR3, CDR3, FR4. Together, the variable regions of the heavy and light chain polypeptides contain or form a binding domain that interacts with/binds an antigen.

It is to be understood that when the term “antibody” is referred to herein, this term is also generally meant to embrace antigen-binding fragments of such antibodies or immunoglobulins, so as to avoid excessive repetition of the associated phrase “antigen-binding fragments” whenever the term “antibodies” or “immunoglobulins” is mentioned. Thus, the present technology encompasses not only antibodies directed against an antigen derived from a taste receptor, olfactory receptor, or an agonist ligand of either receptor, but also fragments of such antibodies which bind these antigens, as described further herein. In embodiments, such antigen-binding fragments are capable of binding the taste receptor, olfactory receptor, or an agonist ligand of either receptor in a manner similar to that of the intact antibody.

The term “antigen-binding fragment” of an antibody as used herein, refers to one or more portions of an antibody that retain the ability to specifically bind to an antigen (e.g., taste receptor, olfactory receptor, or an agonist ligand of either receptor, etc.) or to epitopic regions of an antigen. It has been shown that the antigen-binding function of an antibody can be performed by fragments of a full-length antibody. In an embodiment, the monoclonal antibody fragments function in a manner similar to the intact counterpart monoclonal antibodies. In an embodiment, the monoclonal antibody fragments cross-react with the intact counterpart monoclonal antibodies. In an embodiment, the monoclonal antibody fragments can be used interchangeably with the intact counterpart monoclonal antibodies. Examples of binding fragments encompassed within the term “antigen-binding fragment” of an antibody include (i) a Fab fragment, a monovalent fragment consisting of the V_{L}, V_{H}, C_{L}, and C_{H}1 domains; (ii) a F(ab′)₂ fragment, a bivalent fragment including two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546) which consists of a V_{H} domain; and (vi) an isolated complementarity determining region (CDR). Furthermore, although the two domains of the Fv fragment, V_{L} and V_{H}, are encoded by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), as well as by other techniques known to those having skill in the art. The fragments are screened for activity or utility in the same manner as are intact antibodies.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv including only three CDRs specific for an antigen) has the ability to recognize and bind antigen, although at a lower affinity than the entire binding site.

The Fab fragment also contains the constant domain of the light chain and the first constant domain (CHI) of the heavy chain. Fab fragments differ from Fab′ fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “sFv” antibody fragments comprise the VH and VL domains of antibody, wherein these domains are present in a single polypeptide chain. Preferably, the Fv polypeptide further includes a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., SpringerVerlag, New York, pp. 269-315 (1994).

As noted, the antibodies described herein include those that specifically bind to an epitope on a taste receptor olfactory receptor. To determine an epitope, one can use standard epitope mapping methods known in the art. For example, fragments (peptides) of the taste receptor or olfactory receptor (preferably synthetic peptides) that bind an antibody can be used to determine whether a candidate antibody or antigen-binding fragment thereof binds the same epitope. For linear epitopes, overlapping peptides of a defined length (e.g., 8 or more amino acids) are synthesized. The peptides preferably are offset by 1 amino acid, such that a series of peptides covering every 8 amino acid fragment of the taste receptor or olfactory receptor sequence are prepared. Fewer peptides can be prepared by using larger offsets, e.g., 2 or 3 amino acids. In addition, longer peptides (e.g., 9-, 10- or 11-mers) can be synthesized. Binding of peptides to antibodies can be determined using standard methodologies including surface plasmon resonance (e.g., Biacore) and ELISA assays. For examination of conformational epitopes, which the antibodies provided herein may, in some embodiments, bind, larger peptide fragments can be used. Other methods that use mass spectrometry to define conformational epitopes have been described and can be used (see, e.g., Baerga-Ortiz et al., Protein Science 11:1300-1308, 2002 and references cited therein). Still other methods for epitope determination are provided in standard laboratory reference works, such as Unit 6.8 (“Phage Display Selection and Analysis of B-cell Epitopes”) and Unit 9.8 (“Identification of Antigenic Determinants Using Synthetic Peptide Combinatorial Libraries”) of Current Protocols in Immunology, Coligan et al., eds., John Wiley & Sons. Epitopes can be confirmed by introducing one or more point mutations or deletions into a known epitope, and then testing binding with one or more antibodies to determine which mutations reduce binding of the antibodies.

The antibodies or antigen-binding fragments provided herein may specifically bind a taste receptor or an olfactory receptor with sub-nanomolar affinity. The antibodies or antigen-binding fragments may have binding affinities of about 1×10⁻⁹M or less, about 1×10⁻¹⁰M or less, or about 1×10⁻¹¹M or less. In a particular embodiment, the binding affinity is less than about 5×10⁻¹⁰M.

The antibodies or antigen-binding fragments may have an on rate constant (Kon) to a taste receptor or an olfactory receptor of at least 10²M⁻¹s⁻¹; at least 10³M⁻¹s⁻¹; at least 10⁴M⁻¹s⁻¹; at least 10⁵M⁻¹s⁻¹; at least 10⁶M⁻¹ s⁻¹; or at least 10⁷M⁻¹s⁻¹, as measured by surface plasmon resonance. The antibodies or antigen-binding fragments may have an off rate constant (Koff) to a taste receptor or an olfactory receptor of at most 10⁻³s⁻¹; at most 10⁻⁴s⁻¹; at most 10⁻⁵s⁻¹; or at most 10⁻⁶s⁻¹, as measured by surface plasmon resonance. The antibodies or antigen-binding fragments may have a dissociation constant (K_{D}) to a taste receptor or an olfactory receptor of at most 10⁻⁷ M; at most 10⁻⁸ M; at most 10⁻⁹ M; at most 10⁻¹⁰ M; at most 10⁻¹¹ M; at most 10⁻¹² M; or at most 10⁻¹³ M.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step. Antibodies may be purified by methods commonly performed by those having skill in the art, e.g., affinity chromatography, Protein A chromatography, and the like.

Such isolated antibodies may comprise monoclonal antibodies, polyclonal antibodies, chimeric antibodies, human antibodies, humanized antibodies, and antigen binding fragments or portions thereof. Further provided are other forms of antibodies, such as single chain antibodies, recombinantly produced antibodies, bispecific, heterospecific, or multimeric antibodies, diabodies, etc., as further described herein.

The isolated antibodies described herein encompass various antibody (immunoglobulin) heavy and light chain isotypes, such as the heavy chain classes or isotypes IgG1, IgG2, IgG3, IgG4, IgM, IgA1, IgA2, IgAsec, IgD, IgE, and subtypes thereof, e.g., IgG2a, IgG2b; and the light chain isotypes κ and λ, and subtypes thereof. In one embodiment, the isolated antibodies are of the IgG2a or IgG1 κ isotype. As used herein, “isotype” refers to the antibody class (e.g., IgM or IgG1 or λ1) that is encoded by heavy and light chain constant region genes. The antibodies or antigen-binding fragments thereof can be full length or can include only an antigen-binding fragment, such as the antibody constant and/or variable domain of IgG1, IgG2, IgG3, IgG4, IgM, IgA1, IgA2, IgAsec, IgD, or IgE, or can consist of a Fab fragment, a F(ab′)₂ fragment, and a Fv fragment.

As noted, the antibodies described herein can be polyclonal, monoclonal, or a mixture of polyclonal and monoclonal antibodies. The antibodies can be produced by a variety of techniques well known in the art. Procedures for raising polyclonal antibodies are well known. As a nonlimiting example, polyclonal antibodies are raised by administering a polypeptide from a taste receptor or olfactory receptor subcutaneously to New Zealand white rabbits which have first been bled to obtain pre-immune serum. The polypeptide from the taste receptor or olfactory receptor can be injected at a total volume of 100 μl per site at six different sites, typically with one or more adjuvants. The rabbits are then bled two weeks after the first injection and periodically boosted with the same antigen three times every six weeks. A sample of serum is collected 10 days after each boost. Polyclonal antibodies are recovered from the serum, preferably by affinity chromatography using toxin A and/or toxin B to capture the antibody. This and other procedures for raising polyclonal antibodies are described in Harlow, E. and Lane, D., Eds., Antibodies: A Laboratory Manual (1988), Cold Spring Harbor Laboratory Press, Cold Spring Harbor, N.Y.

Monoclonal antibody production may be effected by techniques which are also well known in the art. The term “monoclonal antibody”, as used herein, refers to a preparation of antibody molecules of single molecular composition. A monoclonal antibody displays a single binding specificity and affinity for a particular epitope of a given antigen or immunogen. The process of monoclonal antibody production involves obtaining immune somatic cells with the potential for producing antibody, in particular B lymphocytes, which have been previously immunized with the antigen of interest either in vivo or in vitro or both, and that are suitable for fusion with a B-cell myeloma line. Monoclonal antibodies can be produced using immune cells and myeloma cells from different species, such as murine and human cells and cell lines, or for example, in mouse strains which have been genetically engineered to harbor a human immune system, as further described below.

In producing antibodies, including polyclonal and monoclonal antibodies, adjuvants may be employed. Nonlimiting examples of adjuvants that are suitable for use include incomplete Freund's adjuvant, aluminum phosphate, aluminum hydroxide, Ribi (i.e., monophosphoryl lipid A, trehalose dimycolate, Mycobacterium cell wall skeleton, and Tween® 80, with 2% squalene), saponins, Quil A, or alum. A cytotoxic T lymphocyte (CTL) response can be primed by conjugating toxins (or fragments, inactive derivatives or analogs thereof) to lipids, such as, for example, tripalmitoyl-S-glycerylcysteinyl-seryl-serine.

In other embodiments, additional immunization methods can be utilized for generating monoclonal antibodies directed against a taste receptor or an olfactory receptor. For example, in vivo immunization of animals (e.g., mice) can be carried out with the desired type and amount of protein or polypeptide, e.g., derived from a taste receptor or olfactory receptor. Such immunizations are repeated as necessary at intervals of up to several weeks to obtain a sufficient titer of antibodies. Once immunized, animals can be used as a source of antibody-producing lymphocytes. Following the last antigen boost, the animals are sacrificed and spleen cells removed. The BALB/c mouse strain is suitable. However, other mouse strains, rabbit, hamster, sheep, goat and frog may also be used as hosts for preparing antibody-producing cells. See Goding (in Monoclonal Antibodies: Principles and Practice, 2d ed., pp. 60-61, Orlando, Fla., Academic Press, 1986). In particular, mouse strains that have human immunoglobulin genes inserted in the genome (and which cannot produce mouse immunoglobulins) can be used. Examples include the HuMAb mouse strains produced by Medarex (now Bristol Myers Squibb)/GenPharm International, and the XenoMouse strains produced by Abgenix. Such mice produce fully human immunoglobulin molecules in response to immunization.

Those antibody-producing cells that are in the dividing plasmablast stage fuse preferentially. Somatic cells may be obtained from, for example, the lymph nodes, spleens, and peripheral blood of antigen-primed animals, and the lymphatic cells of choice depend to a large extent on their empirical usefulness in the particular fusion system. The antibody-secreting lymphocytes are then fused with (mouse) B cell myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, subcloned and grown either in vivo (as ascites) or in vitro to produce large quantities of antibody. Descriptions of hybridoma methodology and technology may be found in Kohler and Milstein, Nature 256:495 (1975) or Harlow, E. and Lane, D., Eds., Antibodies: A Laboratory Manual (1988), Cold Spring Harbor Laboratory Press, Cold Spring Harbor, N.Y.

Alternatively, human somatic cells capable of producing antibody, specifically B lymphocytes, are suitable for fusion with myeloma cell lines. While B lymphocytes from biopsied spleens, tonsils or lymph nodes of an individual may be used, the more easily accessible peripheral blood B lymphocytes (PBLs) are preferred. In addition, human B cells may be directly immortalized by the Epstein-Barr virus (Cole et al., 1995, Monoclonal Antibodies and Cancer Therapy, Alan R. Liss, Inc., pp. 77-96). Although somatic cell hybridization procedures are preferred, in principle, other techniques for producing monoclonal antibodies can be employed, such as viral or oncogenic transformation of B lymphocytes.

Myeloma cell lines suited for use in hybridoma-producing fusion procedures preferably are non-antibody-producing, have high fusion efficiency, and enzyme deficiencies that render them incapable of growing in certain selective media that support the growth of the desired hybridomas. Examples of such myeloma cell lines that may be used for the production of fused cell lines include P3-X63/Ag8, X63-Ag8.653, NS1/1.Ag 4.1, Sp2/0-Ag14, FO, NSO/U, MPC-11, MPC11-X45-GTG 1.7, S194/5XX0 Bul, derived from mice; R210.RCY3, Y3-Ag 1.2.3, IR983F and 4B210 derived from rats; and U-266, GM1500-GRG2, LICR-LON-HMy2, UC729-6, derived from humans (Goding, in Monoclonal Antibodies: Principles and Practice, 2d ed., pp. 65-66, Orlando, Fla., Academic Press, 1986; Campbell, in Monoclonal Antibody Technology, Laboratory Techniques in Biochemistry and Molecular Biology Vol. 13, Burden and Von Knippenberg, eds. pp. 75-83, Amsterdam, Elseview, 1984).

Fusion with mammalian myeloma cells or other fusion partners capable of replicating indefinitely in cell culture is effected by standard and well-known techniques, for example, by using polyethylene glycol (“PEG”) or other fusing agents. See Milstein and Kohler, Eur. J. Immunol. 6:511 (1976).

In other embodiments, the antibodies can be recombinant antibodies. The term “recombinant antibody”, as used herein, is intended to include antibodies that are prepared, expressed, created or isolated by recombinant means, such as antibodies isolated from an animal (e.g., a mouse) that is transgenic for another species' immunoglobulin genes, antibodies expressed using a recombinant expression vector transfected into a host cell, antibodies isolated from a recombinant, combinatorial antibody library, or antibodies prepared, expressed, created, or isolated by any other means that involves splicing of immunoglobulin gene sequences to other DNA sequences.

In yet other embodiments, the antibodies can be chimeric or humanized antibodies. As used herein, the term “chimeric antibody” refers to an antibody that combines a murine immunoglobulin (Ig) variable or hypervariable regions with a human Ig constant region or constant and variable framework regions. In some embodiments, the chimeric antibody includes the variable region of any of the deposited antibodies provided herein and a human constant region. In some embodiments, the human constant region is an human IgG constant region, such as an human IgG1 constant region. The chimeric antibodies can be produced by any method known to those of skill in the art. As used herein, the term “humanized antibody” refers to an antibody that retains substantially only the antigen-binding CDRs from the parent antibody, e.g., murine monoclonal antibody, in association with human framework regions (see, e.g., Waldmann, 1991, Science 252:1657). Such chimeric or humanized antibodies, which retain the binding specificity of the murine antibody, but have human Ig constant/framework regions, are expected to have reduced immunogenicity when administered in vivo. Therefore, the chimeric and humanized antibodies preferably retain the taste or olfactory receptor-neutralizing activities of the monoclonal antibodies provided and are suitable for consumption (e.g., in humans).

The sequences of the humanized mAbs can be designed by the following illustrative, non-limiting method. First, the framework amino acid residues important for the CDR structure are identified. In parallel, human V_{H} and V_{L} sequences having high homology to the murine V_{H} and V_{L}, respectively, are selected from among known human immunoglobulin (germline) sequences. CDR sequences from the murine mAb, together with framework amino acid residues important for maintaining the structure of the CDRs, are grafted into the selected human framework sequences. In addition, human framework amino acid residues that are found to be atypical in the corresponding V region subgroup are substituted with the typical residues to reduce potential immunogenicity of the resulting humanized mAb. These humanized V_{H} and V_{L} regions are cloned into the expression vectors, e.g., pCON Gamma1 and pCON kappa (Lonza Biologics, Berkshire, UK), respectively. These vectors encode the constant region(s) of the human immunoglobulin heavy and light chain genes. 293T cells can be transiently transfected with these expression vectors using the Effectene system (Qiagen, Valencia, Calif.). Cell supernatants containing secreted chimeric mAb can be collected following transfection, e.g., after three days, and purified using Protein A chromatography. Other expression vectors and host cells may be used to recombinantly produce the described antibodies, as understood by those having skill in the art.

Other methods for humanizing antibodies or antigen-binding fragments are well known in the art and include the methods provided in, for example, U.S. Pat. Nos. 5,585,089; 5,693,761; 5,693,762; and 6,180,370. Antibodies or antigen-binding fragments humanized according to the methods provided in these patents are also provided herein.

According to another embodiment, the monoclonal antibodies described herein can be modified to be in the form of a bispecific antibody, a bifunctional antibody, a multispecific antibody, or a heterofunctional antibody. Nonlimiting examples of bispecific and heterospecific antibodies and procedures for making such antibodies may be found in a number of illustrative publications, for example, published International PCT Application Nos.: WO2009/058383, WO2009/030734, WO2007/093630, and WO2008/024188, published U.S. Patent Application Nos.: 20090060910 and 20070071675, U.S. Pat. Nos. 6,071,517; 7,442,778; 7,235,641; 5,932,448; and 5,292,668. The term “bispecific antibody” is intended to include any agent, e.g., a protein, peptide, or protein or peptide complex, which has two different binding specificities. The term “multispecific antibody” is intended to include any agent, e.g., a protein, peptide, or protein or peptide complex, which has more than two different binding specificities. Accordingly, the antibodies include, but are not limited to, bispecific, trispecific, tetraspecific, and other multispecific antibodies. In one embodiment, the antibodies or antigen-binding fragments of the bispecific or multispecific antibodies are humanized.

The term “bispecific antibodies” further includes diabodies. Diabodies provide therapeutic antibodies having dual specificity and being capable of targeting multiple different epitopes with a single molecule. Diabodies are bivalent, bispecific antibodies in which the V_{H} and V_{L} domains are expressed on a single polypeptide chain, but using a linker that is too short to allow for pairing between the two domains on the same chain, thereby forcing the domains to pair with complementary domains of another chain and creating two antigen-binding sites (see e.g., Holliger, P., et al. (1993) Proc. Natl. Acad. Sci. USA 90:6444-6448; Poijak, R. J., et al. (1994) Structure 2:1121-1123). The two antigen-binding regions of the bispecific antibody are either chemically linked or is expressed by a cell genetically engineered to produce the bispecific antibody. (See generally, Fanger et al., 1995 Drug News & Perspec. 8(3):133-137).

In certain embodiments, the antibodies may be human antibodies. The term “human antibody”, as used herein, is intended to include antibodies having variable and constant Ig regions derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences (referred to herein as “humanized antibodies”). Human antibodies directed against toxin A and/or toxin B can be generated using transgenic mice genetically modified and bred to express components of the human immune system rather than the mouse system.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein. These animals have been genetically modified such that there is a functional deletion in the production of endogenous (e.g., murine) antibodies. The animals are further modified to contain all or a portion of the human germline immunoglobulin gene locus such that immunization of these animals results in the production of fully human antibodies to the antigen of interest. Following immunization of these mice (e.g., XenoMouse (Abgenix), HuMAb mice (Medarex/GenPharm)), monoclonal antibodies are prepared according to standard hybridoma technology. These monoclonal antibodies have human immunoglobulin amino acid sequences and, therefore, will not provoke human anti-mouse antibody (HAMA) responses when administered to humans.

It will also be appreciated that vectors and plasmids can readily be engineered to contain and/or express antibody-encoding nucleic acids. As used herein, a “coding region” refers to a region of a nucleotide sequence that encodes a polypeptide sequence; the coding region can include a region coding for a portion of a protein that is later cleaved off, such as a signal peptide. In some instances, the nucleotide and amino acid sequences may include sequences that encode or that are signal peptides.

The antibodies provided herein can be cloned using the following method, as well as other methods known to those of ordinary skill in the art. As a nonlimiting example, total RNA is generated from hybridoma cells, and cDNA is reverse transcribed using an oligo-dT primer. RNase H can be used to remove RNA to make single-stranded cDNA. Spin column purification can be used to remove free nucleotides. Then, a 3′ poly-dG tail can be added with terminal transferase. PCR amplification can be performed using an oligo-dC primer plus a degenerate primer to the constant region. Approximately, 40 cycles can be performed for robust heavy chain amplification. Direct sequencing of the PCR products can then be performed.

As used herein, “specific binding” refers to antibody binding to an antigen located either on a taste or olfactory receptor or on an antigenic antibody as described herein. Typically, the antibody binds with an affinity that is at least two-fold greater than its affinity for binding to a non-specific antigen (e.g., BSA, casein) other than the receptor-based antigen. In an embodiment, an antibody may bind a linear epitope of the target receptor-based antigen, e.g., a taste receptor or an olfactory receptor. In an embodiment, an antibody may bind a conformational epitope of the target antigen, e.g., a taste receptor or an olfactory receptor.

An “effective amount” of an antibody or an antigen-binding fragment disclosed herein is an amount sufficient to carry out a specifically stated purpose. An “effective amount” may be determined empirically and in a routine manner, in relation to the stated purpose.

As described herein, an edible composition that “modulates” the activity of one or more taste receptors or one or more olfactory receptors by either activating or deactivating such receptors to either increase or decrease activity.

### Freeze Dried Compositions

In some embodiments, the composition that is edible or for intranasal administrations include isolated antibodies that have been freeze-dried (i.e., lyophilized). Techniques for producing freeze-dried antibodies are known in the art and are described, for instance, in U.S. Pat. Nos. 5,262,296; 5,908,826; 6,165,467; and references cited therein. In some embodiments, one or more food-grade additive are combined with the isolated antibody prior to freeze-drying. In preparing a freeze-dried antibody, addition of albumin of a heterologous protein to a solution of a monoclonal antibody before freeze-drying has been described (for example, in Japanese patent applications JP-A 60-146833, 61-78730 and 61-78731, and international PCT publication WO 90/11091). The addition of maltose has also been described in the international PCT publication WO 89/11297.

For the purpose of stabilizing the isolated antibody or for the purpose of pH adjusting, isotonicating and buffering the isolated antibody during freeze-drying, the isolated antibody may be combined with one or more freeze-drying additives selected from the group consisting of a carboxylic acid or its salt, inorganic salt, monosaccharide, disaccharide, sugar alcohol, amino acid, and a gelatin.

Representative examples of a carboxylic acid that may be combined with the isolated antibody prior to freeze-drying include, for example, citric acid, acetic acid, oxalic acid, succinic acid and fumaric acid. In one embodiment, the carboxylic acid is citric acid. Representative salts of a carboxylic acid include, for example, sodium citrate, potassium citrate, sodium acetate, potassium acetate, sodium oxalate, potassium oxalate, sodium succinate, potassium succinate, sodium fumarate and potassium fumarate. In one embodiment, the salts of a carboxylic acid is sodium citrate.

Representative examples of an inorganic salt that may be combined with the isolated antibody prior to freeze-drying include, for example, sodium chloride, potassium chloride and magnesium chloride. In one embodiment, the inorganic salt is sodium chloride.

Representative examples of a monosaccharide that may be combined with the isolated antibody prior to freeze-drying include, for example, glucose, mannose, galactose and fructose. In one embodiment, the monosaccharide is glucose or mannose.

Representative examples of a disaccharide that may be combined with the isolated antibody prior to freeze-drying include, for example, maltose, sucrose and lactose. In one embodiment, the disaccharide is maltose or sucrose.

Representative examples of a sugar alcohol that may be combined with the isolated antibody prior to freeze-drying include, for example, sorbitol and mannitol. In one embodiment, the sugar alcohol is mannitol.

Representative examples of an amino acid that may be combined with the isolated antibody prior to freeze-drying include, for example, glycine, alanine, valine, leucine, isoleucine, tyrosine, phenylalanine, serine, threonine, glutamine, glutamic acid, asparagine, aspartic acid, arginine, lysine, histidine, proline, tryptophan, methionine and cysteine. In one embodiment, the amino acid is glycine or arginine.

Representative gelatins that may be combined with the isolated antibody prior to freeze-drying include, for example, neutral-type and acidic-type gelatins. In one embodiment, the gelatin is a chemically modified gelatins such as oxypolygelatin.

According to another exemplary embodiment, the present technology provides a method for modulating the flavor of food, comprising contacting the food with an effective amount of any one of the edible compositions described herein.

According to another exemplary embodiment, the present technology provides a method for activating one or more taste receptors or one or more olfactory receptors, including contacting the taste receptors or olfactory receptors with an effective amount of a composition described herein.

According to another exemplary embodiment, the present technology provides a method for preventing activation of one or more taste receptors or one or more olfactory receptors, including contacting the taste receptors or olfactory receptors with an effective amount of a composition described herein.

According to another exemplary embodiment, the present technology provides a method for modulating activity of one or more taste receptors or one or more olfactory receptors, including contacting the taste receptors or olfactory receptors with an effective amount of a composition described herein.

The present technology, thus generally described, will be understood more readily by reference to the following prophetic Examples, which are provided by way of illustration and is not intended to be limiting of the present technology.

### EXAMPLES

### Example 1

### Preparing Antibodies

This example illustrates representative procedures for preparing monoclonal antibodies that can specifically bind taste receptors or olfactory receptors.

Techniques for producing the monoclonal antibodies are known in the art and are described, for instance, in Goding (in Monoclonal Antibodies: Principles and Practice, 2d ed., pp. 60-61, Orlando, Fla., Academic Press, 1986), U.S. Pat. No. 7,348,414, and references cited therein. Immunogens that may be employed include purified polypeptides or fragments thereof from taste receptors or olfactory receptors, fusion proteins containing such polypeptides, and cells expressing recombinant forms of such polypeptides on the cell surface. Selection of the immunogen can be made by the skilled artisan without undue experimentation.

Mice, such as Balb/c, are immunized with the an immunogen, where the immunogen is derived either from a taste receptor, olfactory receptor, a polypeptide subunit or fraction of either, or an antigen (e.g., sucrose) that activates a receptor. The immunogen is emulsified in Freund's adjuvant and injected subcutaneously or intraperitoneally in an amount from 1-100 micrograms. Alternatively, the immunogen is emulsified in MPL-TDM adjuvant (Ribi Immunochemical 50 Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-immunogen antibodies.

After a suitable antibody titer is detected, the animals “positive” for antibodies can be injected with a final intravenous injection of a purified immunogen. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against the purified immunogen. Determination of “positive” hybridoma cells secreting the desired monoclonal antibodies against the purified immunogen thereof is within the skill in the art.

The positive hybridoma cells can be injected intraperitoneally into syngeneic Balb/c mice to produce ascites containing the anti-immunogen monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding affinity of the antibody can be employed.

### Example 2

### Freeze-Drying Antibodies

Techniques for freeze-drying (i.e., lyophilizing) antibodies are known in the art and are described, for instance, in U.S. Pat. Nos. 5,262,296; 5,908,826; 6,165,467; and references cited therein. The hybridoma cells of Example 1, producing for example monoclonal antibodies reactive to a polypeptide subunit of a human taste receptor, can be cultured, and the monoclonal antibody can purified from the culture supernatant by ammonium sulfate salting-out, gel filtration with SEPHACRYL S-300 (Pharmacia Co.) and column chromatography with a hydroxyapatite HPLC SEPHAROSE (Pharmacia Co.). The monoclonal antibody as obtained by these methods has a purity of 99% or higher, as analyzed by SDS-polyacrylamide gel electrophoresis and HPLC with a gel filtration column. For the purpose of freeze-drying, the monoclonal anti-body is dissolved in a phosphate-buffered physiological saline (PBS) having an adjusted pH value of 7.4, to have a final concentration of about 0.1 mg/ml. On the other hand, gelatin (high-grade gelatin; Nippi Co., type A (neutral gelatin) and type B (acidic gelatin)) can be added thereto to have a final concentration of 0.001 to 1%. The resulting solution is then put in 2 ml-volume polypropylene cryotubes (Corning Co.) under a sterilized condition, each in an amount of 0.5 ml, and frozen therein at −80° C. These are freeze-dried in vacuo. After drying, the same amount, as that before freeze-drying, of a distilled water for injection can be added to the freeze-dried product so that the product dissolves and antigen-binding activity of the monoclonal antibody in the resulting solution can be confirmed. The freeze-dried antibodies can be added to a seasoning, to be added to cooked foods, or incorporated into a food or beverage during production.

### Example 3

### Sensory Analysis of Antibodies

Techniques for tasting the antibodies and edible compositions described herein are known in the art and are described, for instance, by Tancredi et al., Eur. J. Biochem. 271, 2231-2240 (2004) and references. Appropriate amounts of agonist antibodies selective for sweet taste receptors, as described herein, are dissolved in distilled H₂O to make concentrations similar to 10% (w/w) sucrose. The solutions can be adjusted to pH 6.6 with 0.1 M NaOH or HCl. A taste panel of people can be used, preferably including males and females, and nonsmokers of good health and normal sense of taste. Experiments are carried out with the understanding and written consent of all subjects. The subjects taste three known sweeteners as positive controls, deionized water as the negative control, and solutions of the agonist antibodies described herein, in double blind experiments. The sample volumes may be, for example, 150 μL. All solutions are delivered with a micropipette to the anterior part of the subject's tongue. The subject tastes each sample without any time constrains, spits it out, and rinses with tap water within a 1-min interval. Each subject tastes each sample three times. Between tastings of each sample each subject is asked to score the sweetness of the sample on an arbitrary scale. The scale ranges between ‘barely detectable’, ‘weak’, ‘moderate’, ‘strong’ and ‘very strong’.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent compositions, apparatuses, and methods within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as ‘up to,’ ‘at least,’ ‘greater than,’ ‘less than,’ and the like, include the number recited and refer to ranges which can be subsequently broken down into subranges as discussed above. Finally, as will be understood by one skilled in the art, a range includes each individual member.

While certain embodiments have been illustrated and described, it should be understood that changes and modifications can be made therein in accordance with ordinary skill in the art without departing from the technology in its broader aspects as defined in the following claims.

1. A food or beverage comprising an isolated antagonist antibody or an antigen-binding fragment thereof, which specifically binds a sweet, sour or umami taste receptor or a ligand thereof.

2. The food or beverage of claim 1, wherein the isolated antagonist antibody or antigen-binding fragment thereof prevents activation of the sweet, sour or umami taste receptor upon binding the taste receptor or taste receptor ligand.

3. The food or beverage of claim 1, wherein the sweet, sour or umami taste receptor ligand is a tastant.

4. (canceled)

5. The food or beverage of claim 1, further comprising the sweet, sour or umami taste receptor ligand.

6.-7. (canceled)

8. The food or beverage of claim 1, wherein at least one of the antibodies or antigen-binding fragments is in a humanized form; is in chimeric form; is, or is from, a monoclonal antibody; is, or is from, a single-chain antibody.

9. The food or beverage of claim 1, wherein at least one of the antibodies or antigen-binding fragments is or is from, a monoclonal antibody.

10.-23. (canceled)

24. The food or beverage of claim 1, wherein the food or beverage modulates the activity of one or more sweet, sour or umami taste receptors.

25. The food or beverage of claim 1, wherein the sweet, sour or umami taste receptor is a human taste receptor.

26. The food or beverage of claim 1, wherein the sweet, sour or umami taste receptor is a non-human taste receptor.

27-28. (canceled)

29. The food or beverage of claim 28, wherein the taste receptor is a heterodimer comprising two polypeptide subunits independently selected from the Type 1 (TAS1 sweet) family of polypeptides.

30. The food or beverage of claim 29, wherein each polypeptide subunit from the TAS1 family is independently selected from the group consisting of TAS1R1 (SEQ ID NO:1), TAS1R2 (SEQ ID NO:2), and TAS1R3 (SEQ ID NO:3).

31. The food or beverage of claim 29, wherein the taste receptor comprises TAS1R2 (SEQ ID NO:2) and TAS1R3 (SEQ ID NO:3) polypeptide subunits.

32-35. (canceled)

36. The food or beverage of claim 1, wherein the taste receptor is an umami taste receptor

37. The food or beverage of claim 36, wherein the taste receptor comprises TAS1R2 (SEQ ID NO:2) and TAS1R3 (SEQ ID NO:3) polypeptide subunits.

38. The food or beverage of claim 1, wherein the taste receptor is a sour taste receptor.

39. The food or beverage of claim 38, wherein the sour taste receptor is a polypeptide selected from the group consisting of a hyperpolarization-activated cyclic nucleotide-gated channel, HCN1 (SEQ ID NO:31) and HCN4 (SEQ ID NO:32), the amiloride-sensitive cation channel 1, ACCN1 (SEQ ID NO:33), and the two-pore domain potassium channel, TASK-1 (SEQ ID NO:34) polypeptide.

40. The food or beverage of claim 3, wherein the tastant is selected from the group consisting of a sweet substance, sour substance, and a savory substance.

41. The food or beverage of claim 40, wherein the tastant is an agonist ligand that activates the sweet, sour or umami taste receptor.

42. The food or beverage of claim 40, wherein the sweet substance is selected from the group consisting of glucose, fructose, galactose, sucrose, lactose, stevia, aspartame, sucralose, neotame, acesulfame potassium, and saccharin.

43. The food or beverage of claim 40, wherein the sour substance is selected from the group consisting of dilute hydrochloric acid, tartaric acid, citric acid, and carbonic acid.

44. (canceled)

45. The food or beverage of claim 40, wherein the savory substance is monosodium glutamate (MSG).

46. The food or beverage of claim 1, further comprising a dietary additive.

47. The food or beverage of claim 46, wherein the dietary additive is one or more of a protein, carbohydrate, lipid, vitamin, mineral, sweetening agent, food flavoring agent or enhancer, food color, antimicrobial agent, antioxidant, surface modifying agent and an emulsification agent.

48.-51. (canceled)

52. A method for modulating the flavor of food, comprising contacting the food with an effective amount of the food or beverage of claim 1.

53.-73. (canceled)

74. The food or beverage of claim 5, wherein the taste receptor ligand is a compound having a molecular weight less than 500 Daltons.


SEQUENCE LISTING (34 sequences)

SEQ ID NO: 1 - protein, 841 aa, Homo sapiens [sequence omitted]

SEQ ID NO: 2 - protein, 839 aa, Homo sapiens
MGPRAKTISSLFFLLWVLAEPAENSDFYLPGDYLLGGLFSLHANMKGIVHLNFLQVPMCKEYEVKVIGYNLMQAMRFAVEEINNDSSLLPGVLLGYEIVDVCYISNNVQPVLYFLAHEDNLLPIQEDYSNYISRVVAVIGPDNSESVMTVANFLSLFLLPQITYSAISDELRDKVRFPALLRTTPSADHHIEAMVQLMLHFRWNWIIVLVSSDTYGRDNGQLLGERVARRDICIAFQETLPTLQPNQNMTSEERQRLVTIVDKLQQSTARVVVVFSPDLTLYHFFNEVLRQNFTGAVWIASESWAIDPVLHNLTELRHLGTFLGITIQSVPIPGFSEFREWGPQAGPPPLSRTSQSYTCNQECDNCLNATLSFNTILRLSGERVVYSVYSAVYAVAHALHSLLGCDKSTCTKRVVYPWQLLEEIWKVNFTLLDHQIFFDPQGDVALHLEIVQWQWDRSQNPFQSVASYYPLQRQLKNIQDISWHTINNTIPMSMCSKRCQSGQKKKPVGIHVCCFECIDCLPGTFLNHTEDEYECQACPNNEWSYQSETSCFKRQLVFLEWHEAPTIAVALLAALGFLSTLAILVIFWRHFQTPIVRSAGGPMCFLMLTLLLVAYMVVPVYVGPPKVSTCLCRQALFPLCFTICISCIAVRSFQIVCAFKMASRFPRAYSYWVRYQGPYVSMAFITVLKMVIVVIGMLATGLSPTTRTDPDDPKITIVSCNPNYRNSLLFNTSLDLLLSVVGFSFAYMGKELPTNYNEAKFITLSMTFYFTSSVSLCTFMSAYSGVLVTIVDLLVTVLNLLAISLGYFGPKCYMILFYPERNTPAYFNSMIQGYTMRRD

SEQ ID NO: 3 - protein, 852 aa, Homo sapiens
MLGPAVLGLSLWALLHPGTGAPLCLSQQLRMKGDYVLGGLFPLGEAEEAGLRSRTRPSSPVCTRFSSNGLLWALAMKMAVEEINNKSDLLPGLRLGYDLFDTCSEPVVAMKPSLMFLAKAGSRDIAAYCNYTQYQPRVLAVIGPHSSELAMVTGKFFSFFLMPQVSYGASMELLSARETFPSFFRTVPSDRVQLTAAAELLQEFGWNWVAALGSDDEYGRQGLSIFSALAAARGICIAHEGLVPLPRADDSRLGKVQDVLHQVNQSSVQVVLLFASVHAAHALFNYSISSRLSPKVWVASEAWLTSDLVMGLPGMAQMGTVLGFLQRGAQLHEFPQYVKTHLALATDPAFCSALGEREQGLEEDVVGQRCPQCDCITLQNVSAGLNHHQTFSVYAAVYSVAQALHNTLQCNASGCPAQDPVKPWQLLENMYNLTFHVGGLPLRFDSSGNVDMEYDLKLWVWQGSVPRLHDVGRFNGSLRTERLKIRWHTSDNQKPVSRCSRQCQEGQVRRVKGFHSCCYDCVDCEAGSYRQNPDDIACTFCGQDEWSPERSTRCFRRRSRFLAWGEPAVLLLLLLLSLALGLVLAALGLFVHHRDSPLVQASGGPLACFGLVCLGLVCLSVLLFPGQPSPARCLAQQPLSHLPLTGCLSTLFLQAAEIFVESELPLSWADRLSGCLRGPWAWLVVLLAMLVEVALCTWYLVAFPPEVVTDWHMLPTEALVHCRTRSWVSFGLAHATNATLAFLCFLGTFLVRSQPGRYNRARGLTFAMLAYFITWVSFVPLLANVQVVLRPAVQMGALLLCVLGILAAFHLPRCYLLMRQPGLNTPEFFLGGGPGDAQGQNDGNTGNQGKHE

SEQ ID NO: 4 - protein, 299 aa, Homo sapiens
MLESHLIIYFLLAVIQFLLGIFTNGIIVVVNGIDLIKHRKMAPLDLLLSCLAVSRIFLQLFIFYVNVIVIFFIEFIMCSANCAILLFINELELWLATWLGVFYCAKVASVRHPLFIWLKMRISKLVPWMILGSLLYVSMICVFHSKYAGFMVPYFLRKFFSQNATIQKEDTLAIQIFSFVAEFSVPLLIFLFAVLLLIFSLGRHTRQMRNTVAGSRVPGRGAPISALLSILSFLILYFSHCMIKVFLSSLKFHIRRFIFLFFILVIGIYPSGHSLILILGNPKLKQNAKKFLLHSKCCQ

SEQ ID NO: 5 - protein, 316 aa, Homo sapiens
MMGLTEGVFLILSGTQFTLGILVNCFIELVNGSSWFKTKRMSLSDFIITTLALLRIILLCIILTDSFLIEFSPNTHDSGIIMQIIDVSWTFTNHLSIWLATCLGVLYCLKIASFSHPTFLWLKWRVSRVMVWMLLGALLLSCGSTASLINEFKLYSVFRGIEATRNVTEHFRKKRSEYYLIHVLGTLWYLPPLIVSLASYSLLIFSLGRHTRQMLQNGTSSRDPTTEAHKRAIRIILSFFFLFLLYFLAFLIASFGNFLPKTKMAKMIGEVMTMFYPAGHSFILILGNSKLKQTFVVMLRCESGHLKPGSKGPIFS

SEQ ID NO: 6 - protein, 299 aa, Homo sapiens
MLRLFYFSAIIASVILNFVGIIMNLFITVVNCKTWVKSHRISSSDRILFSLGITRFLMLGLFLVNTIYFVSSNTERSVYLSAFFVLCFMFLDSSSVWFVTLLNILYCVKITNFQHSVFLLLKRNISPKIPRLLLACVLISAFTTCLYITLSQASPFPELVTTRNNTSFNISEGILSLVVSLVLSSSLQFIINVTSASLLIHSLRRHIQKMQKNATGFWNPQTEAHVGAMKLMVYFLILYIPYSVATLVQYLPFYAGMDMGTKSICLIFATLYSPGHSVLIIITHPKLKTTAKKILCFKK

SEQ ID NO: 7 - protein, 299 aa, Homo sapiens
MLSAGLGLLMLVAVVEFLIGLIGNGSLVVWSFREWIRKFNWSSYNLIILGLAGCRFLLQWLIILDLSLFPLFQSSRWLRYLSIFWVLVSQASLWFATFLSVFYCKKITTFDRPAYLWLKQRAYNLSLWCLLGYFIINLLLTVQIGLTFYHPPQGNSSIRYPFESWQYLYAFQLNSGSYLPLVVFLVSSGMLIVSLYTHHKKMKVHSAGRRDVRAKAHITALKSLGCFLLLHLVYIMASPFSITSKTYPPDLTSVFIWETLMAAYPSLHSLILIMGIPRVKQTCQKILWKTVCARRCWGP

SEQ ID NO: 8 - protein, 318 aa, Homo sapiens
MADKVQTTLLFLAVGEFSVGILGNAFIGLVNCMDWVKKRKIASIDLILTSLAISRICLLCVILLDCFILVLYPDVYATGKEMRIIDFFWTLTNHLSIWFATCLSIYYFFKIGNFFHPLFLWMKWRIDRVISWILLGCVVLSVFISLPATENLNADFRFCVKAKRKTNLTWSCRVNKTQHASTKLFLNLATLLPFCVCLMSFFLLILSLRRHIRRMQLSATGCRDPSTEAHVRALKAVISFLLLFIAYYLSFLIATSSYFMPETELAVIFGESIALIYPSSHSFILILGNNKLRHASLKVIWKVMSILKGRKFQQHKQI

SEQ ID NO: 9 - protein, 309 aa, Homo sapiens
MFSPADNIFIILITGEFILGILGNGYIALVNWIDWIKKKKISTVDYILTNLVIARICLISVMVVNGIVIVLNPDVYTKNKQQIVIFTFWTFANYLNMWITTCLNVFYFLKIASSSHPLFLWLKWKIDMVVHWILLGCFAISLLVSLIAAIVLSCDYRFHAIAKHKRNITEMFHVSKIPYFEPLTLFNLFAIVPFIVSLISFFLLVRSLWRHTKQIKLYATGSRDPSTEVHVRAIKTMTSFIFFFFLYYISSILMTFSYLMTKYKLAVEFGEIAAILYPLGHSLILIVLNNKLRQTFVRMLTCRKIACVI

SEQ ID NO: 10 - protein, 312 aa, Homo sapiens [sequence omitted]

SEQ ID NO: 11 - protein, 307 aa, Homo sapiens
MLRVVEGIFIFVVVSESVFGVLGNGFIGLVNCIDCAKNKLSTIGFILTGLAISRIFLIWIIITDGFIQIFSPNIYASGNLIEYISYFWVIGNQSSMWFATSLSIFYFLKIANFSNYIFLWLKSRTNMVLPFMIVFLLISSLLNFAYIAKILNDYKMKNDTVWDLNMYKSEYFIKQILLNLGVIFFFTLSLITCIFLIISLWRHNRQMQSNVTGLRDSNTEAHVKAMKVLISFIILFILYFIGMAIEISCFTVRENKLLLMFGMTTTAIYPWGHSFILILGNSKLKQASLRVLQQLKCCEKRKNLRVT

SEQ ID NO: 12 - protein, 300 aa, Homo sapiens
MESALPSIFTLVIIAEFIIGNLSNGFIVLINCIDWVSKRELSSVDKLLIILAISRIGLIWEILVSWFLALHYLAIFVSGTGLRIMIFSWIVSNHFNLWLATIFSIFYLLKIASFSSPAFLYLKWRVNKVILMILLGTLVFLFLNLIQINMHIKDWLDRYERNTTWNFSMSDFETFSVSVKFTMTMFSLTPFTVAFISFLLLIFSLQKHLQKMQLNYKGHRDPRTKVHTNALKIVISFLLFYASFFLCVLISWISELYQSTVIYMLCETIGVFSPSSHSFLLILGNAKLRQAFLLVAAKVW

SEQ ID NO: 13 - protein, 317 aa, Homo sapiens
MGGVIKSIFTFVLIVEFIIGNLGNSFIALVNCIDWVKGRKISSVDRILTALAISRISLVWLIFGSWCVSVFFPALFATEKMFRMLTNIWTVINHFSVWLATGLGTFYFLKIANFSNSIFLYLKWRVKKVVLVLLLVTSVFLFLNIALINIHINASINGYRRNKTCSSDSSNFTRFSSLIVLTSTVFIFIPFTLSLAMFLLLIFSMWKHRKKMQHTVKISGDASTKAHRGVKSVITFFLLYAIFSLSFFISVWTSERLEENLIILSQVMGMAYPSCHSCVLILGNKKLRQASLSVLLWLRYMFKDGEPSGHKEFRESS

SEQ ID NO: 14 - protein, 291 aa, Homo sapiens
MIPIQLTVFFMIIYVLESLTIIVQSSLIVAVLGREWLQVRRLMPVDMILISLGISRFCLQWASMLNNFCSYFNLNYVLCNLTITWEFFNILTFWLNSLLTVFYCIKVSSFTHHIFLWLRWRILRLFPWILLGSLMITCVTIIPSAIGNYIQIQLLTMEHLPRNSTVTDKLENFHQYQFQAHTVALVIPFILFLASTIFLMASLTKQIQHHSTGHCNPSMKARFTALRSLAVLFIVFTSYFLTILITIIGTLFDKRCWLWVWEAFVYAFILMHSTSLMLSSPTLKRILKGKC

SEQ ID NO: 15 - protein, 299 aa, Homo sapiens
MMCFLLIISSILVVFAFVLGNVANGFIALVNVIDWVNTRKISSAEQILTALVVSRIGLLWVMLFLWYATVFNSALYGLEVRIVASNAWAVTNHFSMWLAASLSIFCLLKIANFSNLISLHLKKRIKSVVLVILLGPLVFLICNLAVITMDERVWTKEYEGNVTWKIKLRNAIHLSSLTVTTLANLIPFTLSLICFLLLICSLCKHLKKMRLHSKGSQDPSTKVHIKALQTVTSFLMLFAIYFLCIITSTWNLRTQQSKLVLLLCQTVAIMYPSFHSFILIMGSRKLKQTFLSVLWQMTR

SEQ ID NO: 16 - protein, 309 aa, Homo sapiens
MMSFLHIVFSILVVVAFILGNFANGFIALINFIAWVKRQKISSADQIIAALAVSRVGLLWVILLHWYSTVLNPTSSNLKVIIFISNAWAVTNHFSIWLATSLSIFYLLKIVNFSRLIFHHLKRKAKSVVLVIVLGSLFFLVCHLVMKHTYINVWTEECEGNVTWKIKLRNAMHLSNLTVAMLANLIPFTLTLISFLLLIYSLCKHLKKMQLHGKGSQDPSTKIHIKALQTVTSFLILLAIYFLCLIISFWNFKMRPKEIVLMLCQAFGIIYPSFHSFILIWGNKTLKQTFLSVLWQVTCWAKGQNQSTP

SEQ ID NO: 17 - protein, 319 aa, Homo sapiens
MITFLPIIFSILIVVIFVIGNFANGFIALVNSIEWVKRQKISFVDQILTALAVSRVGLLWVLLLHWYATQLNPAFYSVEVRITAYNVWAVTNHFSSWLATSLSMFYLLRIANFSNLIFLRIKRRVKSVVLVILLGPLLFLVCHLFVINMDETVWTKEYEGNVTWKIKLRSAMYHSNMTLTMLANFVPLTLTLISFLLLICSLCKHLKKMQLHGKGSQDPSTKVHIKALQTVTSFLLLCAIYFLSMIISVCNFGRLEKQPVFMFCQAIIFSYPSTHPFILILGNKKLKQIFLSVLRHVRYWVKDRSLRLHRFTRGALCVF

SEQ ID NO: 18 - protein, 309 aa, Homo sapiens
MTTFIPIIFSSVVVVLFVIGNFANGFIALVNSIERVKRQKISFADQILTALAVSRVGLLWVLLLNWYSTVFNPAFYSVEVRTTAYNVWAVTGHFSNWLATSLSIFYLLKIANFSNLIFLHLKRRVKSVILVMLLGPLLFLACQLFVINMKEIVRTKEYEGNLTWKIKLRSAVYLSDATVTTLGNLVPFTLTLLCFLLLICSLCKHLKKMQLHGKGSQDPSTKVHIKALQTVIFFLLLCAVYFLSIMISVWSFGSLENKPVFMFCKAIRFSYPSIHPFILIWGNKKLKQTFLSVLRQVRYWVKGEKPSSP

SEQ ID NO: 19 - protein, 333 aa, Homo sapiens
  MOD_RES (49)..(49): Any amino acid
MLTLTRIRTVSYEVRSTFLFISVLEFAVGFLTNAFVFLVNFWDVVKRQXLSNSDCVLLCLSISRLFLHGLLFLSAIQLTHFQKLSEPLNHSYQAIIMLWMIANQANLWLAACLSLLYCSKLIRFSHTFLICLASWVSRKISQMLLGIILCSCICTVLCVWCFFSRPHFTVTTVLFMNNNTRLNWQIKDLNLFYSFLFCYLWSVPPFLLFLVSSGMLTVSLGRHMRTMKVYTRNSRDPSLEAHIKALKSLVSFFCFFVISSCAAFISVPLLILWRDKIGVMVCVGIMAACPSGHAAVLISGNAKLRRAVMTILLWAQSSLKVRADHKADSRTLC

SEQ ID NO: 20 - protein, 338 aa, Homo sapiens
MLGRCFPPDTKEKQQLRMTKLCDPAESELSPFLITLILAVLLAEYLIGIIANGFIMAIHAAEWVQNKAVSTSGRILVFLSVSRIALQSLMMLEITISSTSLSFYSEDAVYYAFKISFIFLNFCSLWFAAWLSFFYFVKIANFSYPLFLKLRWRITGLIPWLLWLSVFISFSHSMFCINICTVYCNNSFPIHSSNSTKKTYLSEINVVGLAFFFNLGIVTPLIMFILTATLLILSLKRHTLHMGSNATGSNDPSMEAHMGAIKAISYFLILYIFNAVALFIYLSNMFDINSLWNNLCQIIMAAYPASHSILLIQDNPGLRRAWKRLQLRLHLYPKEWTL

SEQ ID NO: 21 - protein, 323 aa, Homo sapiens
MATVNTDATDKDISKFKVTFTLVVSGIECITGILGSGFITAIYGAEWARGKTLPTGDRIMLMLSFSRLLLQIWMMLENIFSLLFRIVYNQNSVYILFKVITVFLNHSNLWFAAWLKVFYCLRIANFNHPLFFLMKRKIIVLMPWLLRLSVLVSLSFSFPLSRDVFNVYVNSSIPIPSSNSTEKKYFSETNMVNLVFFYNMGIFVPLIMFILAATLLILSLKRHTLHMGSNATGSRDPSMKAHIGAIKATSYFLILYIFNAIALFLSTSNIFDTYSSWNILCKIIMAAYPAGHSVQLILGNPGLRRAWKRFQHQVPLYLKGQTL

SEQ ID NO: 22 - protein, 307 aa, Homo sapiens
MQAALTAFFVLLFSLLSLLGIAANGFIVLVLGREWLRYGRLLPLDMILISLGASRFCLQLVGTVHNFYYSAQKVEYSGGLGRQFFHLHWHFLNSATFWFCSWLSVLFCVKIANITHSTFLWLKWRFPGWVPWLLLGSVLISFIITLLFFWVNYPVYQEFLIRKFSGNMTYKWNTRIETYYFPSLKLVIWSIPFSVFLVSIMLLINSLRRHTQRMQHNGHSLQDPSTQAHTRALKSLISFLILYALSFLSLIIDAAKFISMQNDFYWPWQIAVYLCISVHPFILIFSNLKLRSVFSQLLLLARGFWVA

SEQ ID NO: 23 - protein, 314 aa, Homo sapiens
MATELDKIFLILAIAEFIISMLGNVFIGLVNCSEGIKNQKVFSADFILTCLAISTIGQLLVILFDSFLVGLASHLYTTYRLGKTVIMLWHMTNHLTTWLATCLSIFYFFKIAHFPHSLFLWLRWRMNGMIVMLLILSLFLLIFDSLVLEIFIDISLNIIDKSNLTLYLDESKTLYDKLSILKTLLSLTSFIPFSLFLTSLLFLFLSLVRHTRNLKLSSLGSRDSSTEAHRRAMKMVMSFLFLFIVHFFSLQVANGIFFMLWNNKYIKFVMLALNAFPSCHSFILILGNSKLRQTAVRLLWHLRNYTKTPNALPL

SEQ ID NO: 24 - protein, 309 aa, Homo sapiens
MITFLPIIFSSLVVVTFVIGNFANGFIALVNSIEWFKRQKISFADQILTALAVSRVGLLWVLLLNWYSTVLNPAFNSVEVRTTAYNIWAVINHFSNWLATTLSIFYLLKIANFSNFIFLHLKRRVKSVILVMLLGPLLFLACHLFVINMNEIVRTKEFEGNMTWKIKLKSAMYFSNMTVTMVANLVPFTLTLLSFMLLICSLCKHLKKMQLHGKGSQDPSTKVHIKALQTVISFLLLCAIYFLSIMISVWSFGSLENKPVFMFCKAIRFSYPSIHPFILIWGNKKLKQTFLSVFWQMRYWVKGEKTSSP

SEQ ID NO: 25 - protein, 309 aa, Homo sapiens
MTTFIPIIFSSVVVVLFVIGNFANGFIALVNSIERVKRQKISFADQILTALAVSRVGLLWVLLLNWYSTVFNPAFYSVEVRTTAYNVWAVTGHFSNWLATSLSIFYLLKIANFSNLIFLHLKRRVKSVILVMLLGPLLFLACQLFVINMKEIVRTKEYEGNMTWKIKLRSAVYLSDATVTTLGNLVPFTLTLLCFLLLICSLCKHLKKMQLHGKGSQDPSTKVHIKALQTVIFFLLLCAVYFLSIMISVWSFGSLENKPVFMFCKAIRFSYPSIHPFILIWGNKKLKQTFLSVLRQVRYWVKGEKPSSP

SEQ ID NO: 26 - protein, 299 aa, Homo sapiens [sequence omitted]

SEQ ID NO: 27 - protein, 309 aa, Homo sapiens
MITFLPIIFSILIVVTFVIGNFANGFIALVNSIEWFKRQKISFADQILTALAVSRVGLLWVLVLNWYATELNPAFNSIEVRITAYNVWAVINHFSNWLATSLSIFYLLKIANFSNLIFLHLKRRVKSVVLVILLGPLLFLVCHLFVINMNQIIWTKEYEGNMTWKIKLRSAMYLSNTTVTILANLVPFTLTLISFLLLICSLCKHLKKMQLHGKGSQDPSMKVHIKALQTVTSFLLLCAIYFLSIIMSVWSFESLENKPVFMFCEAIAFSYPSTHPFILIWGNKKLKQTFLSVLWHVRYWVKGEKPSSS

SEQ ID NO: 28 - protein, 309 aa, Homo sapiens [sequence omitted]

SEQ ID NO: 29 - protein, 299 aa, Homo sapiens
MITFLYIFFSILIMVLFVLGNFANGFIALVNFIDWVKRKKISSADQILTALAVSRIGLLWALLLNWYLTVLNPAFYSVELRITSYNAWVVTNHFSMWLAANLSIFYLLKIANFSNLLFLHLKRRVRSVILVILLGTLIFLVCHLLVANMDESMWAEEYEGNMTGKMKLRNTVHLSYLTVTTLWSFIPFTLSLISFLMLICSLCKHLKKMQLHGEGSQDLSTKVHIKALQTLISFLLLCAIFFLFLIVSVWSPRRLRNDPVVMVSKAVGNIYLAFDSFILIWRTKKLKHTFLLILCQIRC

SEQ ID NO: 30 - protein, 318 aa, Homo sapiens
MNGDHMVLGSSVTDKKAIILVTILLLLRLVAIAGNGFITAALGVEWVLRRMLLPCDKLLVSLGASRFCLQSVVMGKTIYVFLHPMAFPYNPVLQFLAFQWDFLNAATLWSSTWLSVFYCVKIATFTHPVFFWLKHKLSGWLPWMLFSSVGLSSFTTILFFIGNHRMYQNYLRNHLQPWNVTGDSIRSYCEKFYLFPLKMITWTMPTAVFFICMILLITSLGRHRKKALLTTSGFREPSVQAHIKALLALLSFAMLFISYFLSLVFSAAGIFPPLDFKFWVWESVIYLCAAVHPIILLFSNCRLRAVLKSRRSSRCGTP

SEQ ID NO: 31 - protein, 890 aa, Homo sapiens
MEGGGKPNSSSNSRDDGNSVFPAKASATGAGPAAAEKRLGTPPGGGGAGAKEHGNSVCFKVDGGGGGGGGGGGGEEPAGGFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILNELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRRAFETVAIDRLDRIGKKNSILLQKFQKDLNTGVFNNQENEILKQIVKHDREMVQAIAPINYPQMTTLNSTSSTTTPTSRMRTQSPPVYTATSLSHSNLHSPSPSTQTPQPSAILSPCSYTTAVCSPPVQSPLAARTFHYASPTASQLSLMQQQPQQQVQQSQPPQTQPQQPSPQPQTPGSSTPKNEVHKSTQALHNTNLTREVRPLSASQPSLPHEVSTLISRPHPTVGESLASIPQPVTAVPGTGLQAGGRSTVPQRVTLFRQMSSGAIPPNRGVPPAPPPPAAALPRESSSVLNTDPDAEKPRFASNL

SEQ ID NO: 32 - protein, 1203 aa, Homo sapiens
MDKLPPSMRKRLYSLPQQVGAKAWIMDEEEDAEEEGAGGRQDPSRRSIRLRPLPSPSPSAAAGGTESRSSALGAADSEGPARGAGKSSTNGDCRRFRGSLASLGSRGGGSGGTGSGSSHGHLHDSAEERRLIAEGDASPGEDRTPPGLAAEPERPGASAQPAASPPPPQQPPQPASASCEQPSVDTAIKVEGGAAAGDQILPEAEVRLGQAGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFMVDFISSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPDDCWVSINNMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLNSGVFNYQENEIIQQIVQHDREMAHCAHRVQAAASATPTPTPVIWTPLIQAPLQAAAATTSVAIALTHHPRLPAAIFRPPPGSGLGNLGAGQTPRHLKRLQSLIPSALGSASPASSPSQVDTPSSSSFHIQQLAGFSAPAGLSPLLPSSSSSPPPGACGSPSAPTPSAGVAATTIAGFGHFHKALGGSLSSSDSPLLTPLQPGARSPQAAQPSPAPPGARGGLGLPEHFLPPPPSSRSPSSSPGQLGQPPGELSLGLATGPLSTPETPPRQPEPPSLVAGASGGASPVGFTPRGGLSPPGHSPGPPRTFPSAPPRASGSHGSLLLPPASSPPPPQVPQRRGTPPLTPGRLTQDLKLISASQPALPQDGAQTLRRASPHSSGESMAAFPLFPRAGGGSGGSGSSGGLGPPGRPYGAIPGQHVTLPRKTSSGSLPPPLSLFGARATSSGGPPLTAGPQREPGARPEPVRSKLPSNL

SEQ ID NO: 33 - protein, 512 aa, Homo sapiens
MDLKESPSEGSLQPSSIQIFANTSTLHGIRHIFVYGPLTIRRVLWAVAFVGSLGLLLVESSERVSYYFSYQHVTKVDEVVAQSLVFPAVTLCNLNGFRFSRLTTNDLYHAGELLALLDVNLQIPDPHLADPSVLEALRQKANFKHYKPKQFSMLEFLHRVGHDLKDMMLYCKFKGQECGHQDFTTVFTKYGKCYMFNSGEDGKPLLTTVKGGTGNGLEIMLDIQQDEYLPIWGETEETTFEAGVKVQIHSQSEPPFIQELGFGVAPGFQTFVATQEQRLTYLPPPWGECRSSEMGLDFFPVYSITACRIDCETRYIVENCNCRMVHMPGDAPFCTPEQHKECAEPALGLLAEKDSNYCLCRTPCNLTRYNKELSMVKIPSKTSAKYLEKKFNKSEKYISENILVLDIFFEALNYETIEQKKAYEVAALLGDIGGQMGLFIGASILTILELFDYIYELIKEKLLDLLGKEEDEGSHDENVSTCDTMPNHSETISHTVNVPLQTTLGTLEEIAC

SEQ ID NO: 34 - protein, 394 aa, Homo sapiens
MKRQNVRTLALIVCTFTYLLVGAAVFDALESEPELIERQRLELRQQELRARYNLSQGGYEELERVVLRLKPHKAGVQWRFAGSFYFAITVITTIGYGHAAPSTDGGKVFCMFYALLGIPLTLVMFQSLGERINTLVRYLLHRAKKGLGMRRADVSMANMVLIGFFSCISTLCIGAAAFSHYEHWTFFQAYYYCFITLTTIGFGDYVALQKDQALQTQPQYVAFSFVYILTGLTVIGAFLNLVVLRFMTMNAEDEKRDAEHRALLTRNGQAGGGGGGGSAHTTDTASSTAAAGGGGFRNVYAEVLHFQSMCSCLWYKSREKLQYSIPMIIPRDLSTSDTCVEQSHSSPGGGGRYSDTPSRRCLCSGAPRSAISSVSTGLHSLSTFRGLMKRRSSV